(19) United States
(12) Patent Application Publication
(10) Pub. No.: US 2025/0129400 A1
(43) Pub. Date: Apr. 24, 2025

(54) COMPOSITIONS AND METHODS FOR PREPARING CAPPED CIRCULAR RNA MOLECULES

(71) Applicants: The Broad Institute, Inc., Cambridge, MA (US); Massachusetts Institute of Technology, Cambridge, MA (US)

(72) Inventors: Xiao WANG, Cambridge, MA (US); Hongyu CHEN, Cambridge, MA (US); Abhishek ADITHAM, Cambridge, MA (US)

(73) Assignees: The Broad Institute, Inc., Cambridge, MA (US); Massachusetts Institute of Technology, Cambridge, MA (US)

(21) Appl. No.: 19/000,110

(22) Filed: Dec. 23, 2024

Related U.S. Application Data
(63) Continuation of application No. PCT/US2023/069110, filed on Jun. 26, 2023.
(60) Provisional application No. 63/355,456, filed on Jun. 24, 2022.
(60) Provisional application No. 63/480,291, filed on Jan. 17, 2023.

(51) Int. Cl. C12P 19/34; C07H 21/02; C12N 9/00; C12N 9/22; C12N 15/11

(52) U.S. Cl. CPC C12P 19/34; C07H 21/02; C12N 9/22; C12N 9/93; C12N 15/11; C12N 2310/122; C12N 2310/321; C12N 2310/3231; C12N 2310/333; C12N 2310/3341; C12Y 605/01001

(57) ABSTRACT

The present disclosure provides compositions, reagents, and methods for producing capped, circular RNA molecules, circularized RNA molecules, and in particular, circularized mRNA molecules encoding a polypeptide such as a therapeutic protein.

### CROSS REFERENCE TO RELATED APPLICATIONS

This application is a continuation of International Application No. PCT/US2023/069110, filed Jun. 26, 2023, which claims priority to U.S. Provisional Application No. 63/355,456, filed Jun. 24, 2022, and U.S. Provisional Application No. 63/480,291, filed Jan. 17, 2023, the disclosures of which are expressly incorporated by reference herein in their entireties.

### INCORPORATION BY REFERENCE OF SEQUENCE LISTING PROVIDED ELECTRONICALLY

This application contains a Sequence Listing submitted as an electronic text file named “114203-1503_SL.xml,” having a size in bytes of 24,183 bytes, and created on Dec. 23, 2024. The information contained in this electronic file is hereby incorporated by reference in its entirety.

### BACKGROUND

RNA therapeutics have recently developed rapidly as a field, as evidenced by recent clinical demonstrations of successful mRNA vaccines against SARS-CoV-2. See, Polack et al., 2020, Safety and Efficacy of the BNT162b2 mRNA Covid-19 Vaccine. N. Engl. J. Med. 383, 2603-2615; Lombardi et al., 2021, Mini Review Immunological Consequences of Immunization With COVID-19 mRNA Vaccines: Preliminary Results. Front. Immunol. 12, 657711. mRNA's inherent programmability and relative ease of production underlie its potential to supplant conventional protein-based therapeutics. See, Sahin et al, 2014, mRNA-based therapeutics-developing a new class of drugs,” Nature reviews Drug discovery 13: 759-780. In addition to demonstrated clinical applications as exemplified by COVID vaccines, mRNA has been used experimentally to express vascular regeneration factors and to generate vaccines against influenza and Zika virus (Zangi et al., 2013, Nature biotechnology 31: 898; Bahl et al., 2017, Molecular Therapy 25: 1316-1327; Richner et al., 2017, Cell 168: 1114-1125).

mRNA is also an emerging therapeutic modality due to its ability to produce proteins of interest (POI) rapidly in vivo. Some major advantages of mRNA as a platform are its programmability, capacity for transience, ease of production, and lack of risk of genomic integration compared to DNA-based therapeutic approaches. In eukaryotic cells, canonical mRNA is linear and contains a 5′ 7-methylguanosine cap (m⁷G) and 3′ poly(A) tail, both of which are indispensable for efficient translation within a cell. Recent studies have described that circular mRNAs (circRNAs) possess an increased half-life in cells, compared to linear mRNAs, because of their reduced susceptibility to exonucleases. However, cap-independent translation by circRNA is not as efficient as the cap-dependent translation by linear RNA.

There thus remains a need in this art for reagents and methods for producing and using compositions comprising circularized RNA molecules and in particular circularized mRNA molecules, encoding polypeptides such as therapeutic proteins, to produce a useful, particularly a therapeutically useful, phenotypic effect on the recipient cells, among other uses.

### SUMMARY OF INVENTION

This invention provides compositions, reagents, and methods comprising RNA molecules preferably encoding a polypeptide, wherein the RNA molecule is a circularized RNA molecule and in particular circularized mRNA molecule.

In certain embodiments, the type 2 capped, circularized RNA molecules comprise a mRNA region encoding the polypeptide, a 5′ end containing a cap structure, a derivatized nucleotide located between the cap structure and the mRNA region; and a 3′ end covalently linked to the derivatized nucleotide.

In certain embodiments, the type 1 capped, circularized RNA molecules comprise an RNA oligonucleotide comprising a 5′ end containing a cap structure and a 3′ end moiety; a circular RNA molecule comprising an mRNA encoding a polypeptide; and a derivatized nucleotide located within the circular RNA molecule, wherein the 3′ end moiety of the oligonucleotide is covalently linked to the derivatized nucleotide on the circular RNA molecule.

In certain alternative embodiments, the type 3 capped, circularized RNA molecules comprise an RNA oligonucleotide comprising a 5′ end containing a cap structure and a 3′ end moiety; a circular RNA molecule comprising twister ribozyme, an mRNA encoding a polypeptide, an oligonucleotide portion that forms a hairpin, and a derivatized nucleotide located within the hairpin, wherein the 3′ end moiety of the oligonucleotide is covalently linked to the derivatized nucleotide within the hairpin of the circular RNA molecule.

In certain embodiments, the derivatized nucleotide comprises a moiety that can react with the 3′ end moiety by bioconjugation chemistry such as click chemistry. In addition, the cap structure includes 7-methylguanosine (m⁷G), 7-benzylguanosine (Bn⁷G), 7-chlorobenzylguanosine (ClBn⁷G), chlorobenzyl-O-ethoxyguanosine (ClBnOEt⁷G), or any derivative thereof. In certain embodiments, the 7-methylguanosine cap structure further comprises one or more Locked Nucleic Acid (LNA), or one or more 2′-methoxy (2OMe), or any derivative thereof.

In certain embodiments, the type 2 and type 1 capped circular RNA molecule comprises one or more modified nucleotides such as pseudouridine, N′-methylpseudouridine (m¹Ψ), 6-methyladenosine (m⁶A), 5-methylcytidine, inosine, or any derivatives thereof. In some embodiments, the modified nucleotides comprise locked nucleic acid (LNA), 2′-methoxyribose (2-OMe), 2-methoxyehthoxy (2-MOE) sugar backbone, or any derivatives thereof.

In certain embodiments, the type 3 capped circular RNA molecule comprises one or more modified nucleotides such as 6-methyladenosine (m⁶A), 5-methylcytidine, inosine, or any derivatives thereof. In certain embodiments, the modified nucleotides comprise locked nucleic acid (LNA), 2′-methoxyribose (2-OMe), 2-methoxyehthoxy (2-MOE) sugar backbone, or any derivatives thereof.

In certain embodiments, the type 1 and type 3 capped circular RNA molecule comprises a circular RNA comprising a plurality of mRNA regions encoding a plurality of polypeptides. In these embodiments, the type 1 and type 3 capped circular RNA molecule can also comprise a plurality of RNA oligonucleotides comprising a 5′ end containing the cap structure and a 3′ end moiety, and a plurality of derivatized nucleotides at a position in the circular RNA 5′ to each of the mRNA regions encoding a peptide or polypeptide, wherein each 3′ end of each of the plurality of the RNA oligonucleotides is covalently linked to each of the plurality of the derivatized nucleotides. In some embodiments, each mRNA region encoding the peptide or the polypeptide comprises a 3′ polyA sequence, wherein the polypeptides encode Cas9, base editors, or derivatives thereof, or therapeutic proteins.

Further provided are pharmaceutical compositions of the capped, circularized RNA molecules provided by the invention, comprising specific embodiments of the capped, circularized RNA molecules provided by the invention and pharmaceutically acceptable adjuvants, excipients, carriers, or diluents.

The invention also provides methods for producing a type 2 capped, circularized RNA molecules of this aspect of the invention, the methods comprising synthesizing an RNA oligonucleotide comprising a 5′ end containing a cap structure, an mRNA encoding a peptide or polypeptide, a derivatized nucleotide located between the cap structure and the mRNA region encoding the polypeptide, and a 3′ end containing moiety; and reacting the derivatized nucleotide with the 3′ end moiety to form the covalently linked capped circular RNA molecule. In certain embodiments, the synthesis of the RNA oligonucleotide comprises the steps of: synthesizing a first RNA oligonucleotide comprising the 5′ end containing a cap structure, the mRNA encoding a peptide or polypeptide, and a hairpin structure between the capped 5′ end and the mRNA encoding the peptide or polypeptide; derivatizing a nucleotide within the hairpin structure of the first RNA; synthesizing a second RNA oligonucleotide comprising a 3′ end moiety reactive with the derivatized nucleotide; and ligating the 3′ end of the first RNA molecule with 5′ end of the second RNA molecule. In certain embodiments, the synthesis of the RNA oligonucleotide comprises the steps of: synthesizing a first RNA oligonucleotide primer comprising the 5′ end containing a cap structure, the derivatized nucleotide, and a complementary sequence to a DNA template encoding a peptide or a polypeptide; transcribing the first RNA oligonucleotide from the primer along the DNA template to produce an mRNA encoding a peptide or a polypeptide; synthesizing a second RNA oligonucleotide comprising a 3′ end containing a moiety; ligating the 3′ end of the first RNA oligonucleotide encoding the peptide or polypeptide sequence with the 5′ end of the second RNA molecule.

The invention also provides methods for producing a type 1 capped, circularized RNA molecules of this aspect of the invention, the methods comprising the steps of: producing a circularized RNA molecule comprising an mRNA region encoding a peptide or polypeptide, and a derivatized nucleotide outside the mRNA region; synthesizing an RNA oligonucleotide comprising a 5′ end containing cap structure and a 3′ end containing a moiety reactive with the derivatized nucleotide; and reacting the derivatized nucleotide with the 3′ end moiety of the RNA oligonucleotide form a covalently link between the RNA oligonucleotide and the circular RNA. In certain embodiments, the the derivatized nucleotide comprises a moiety that can react with the 3′ end moiety by bioconjugation chemistry, wherein in the bioconjugation chemistry is click chemistry. In addition, the circularized RNA is produced by ribozyme-mediated splicing, enzymatic ligation, or click chemistry-mediated circularization.

In certain embodiments, the synthesis of the circular RNA oligonucleotide of type 1 capped, circularized RNA molecules comprises the steps of: synthesizing an RNA oligonucleotide comprising the mRNA region encoding a peptide or polypeptide, and a complementary sequence on 5′ and 3′ ends to facilitate circularization, wherein the derivatized nucleotide is located within the complementary sequence; and circularizing the RNA oligonucleotide. In certain embodiments, the complementary sequence comprises a single cytidine nucleotide, wherein the single cytidine is the derivatized nucleotide. In certain embodiments, the synthesis of the circular RNA oligonucleotide comprises the steps of: synthesizing an RNA oligonucleotide comprising the mRNA region encoding a peptide or polypeptide, and a hairpin structure containing an enzyme-recognition site for introducing the derivatized oligonucleotide in the RNA oligonucleotide; reacting the RNA oligonucleotide with the enzyme to produce the derivatized nucleotide within the hairpin structure; and circularizing the RNA oligonucleotide. In certain embodiments, the synthesis of the circular RNA oligonucleotide comprises the steps of: synthesizing a first RNA oligonucleotide comprising the mRNA region encoding a peptide or polypeptide, and a hydroxyl group on both 5′ and 3′ ends; synthesizing a second RNA oligonucleotide comprising the derivatized nucleotide, and a phosphate on both 5′ and 3′ ends; ligating 5′ phosphate end and 3′ hydroxyl end; and ligating 5′ hydroxyl end and 3′ phosphate end of the first and the second oligonucleotide respectively to produce a circularized RNA oligonucleotide. In certain embodiments, the synthesis of the circular RNA oligonucleotide comprises the steps of: synthesizing a first RNA oligonucleotide comprising the mRNA region encoding a peptide or polypeptide, a 5′ end containing a triphosphate, and a 3′ end containing a hydroxyl; synthesizing a second RNA oligonucleotide comprising the derivatized nucleotide, and a phosphate on both 5′ and 3′ ends; ligating the 3′ end of the first oligonucleotide to the 5′ end of the second oligonucleotide to produce a third oligonucleotide; hydrolyzing the triphosphate on the 5′ end of the third oligonucleotide; and ligating the 5′ end to the 3′ end of the third oligonucleotide to produce a circularized RNA oligonucleotide. In certain embodiments, the synthesis of the circular RNA oligonucleotide comprises the steps of: synthesizing an RNA oligonucleotide primer comprising the derivatized nucleotide, and a complementary sequence to a DNA template encoding a peptide or a polypeptide; transcribing the RNA oligonucleotide to further comprise an mRNA encoding a peptide or a polypeptide; circularizing the RNA oligonucleotide.

The invention also provides methods for producing a type 3 capped, circularized RNA molecules of this aspect of the invention, the methods comprising the steps of: producing a circularized RNA molecule comprising an mRNA region encoding a peptide or polypeptide, and a derivatized nucleotide outside the mRNA region; synthesizing an RNA oligonucleotide comprising a 5′ end containing cap structure and a 3′ end containing a moiety reactive with the derivatized nucleotide; and reacting the derivatized nucleotide with the 3′ end moiety of the RNA oligonucleotide form a covalently link between the RNA oligonucleotide and the circular RNA, wherein the synthesis of the circular RNA oligonucleotide, further comprises the steps of: synthesizing an RNA oligonucleotide comprising the mRNA region encoding a peptide or polypeptide, a hairpin structure containing an enzyme-recognition site, and a twister ribozyme sequence on both 5′ and 3′ ends; reacting the RNA oligonucleotide with the enzyme to produce the derivatized nucleotide within the hairpin structure; circularizing the RNA oligonucleotide using the twister ribozyme sequence.

The capped, circularized RNA molecules provided by the invention advantageously increase stability and translation efficiency of the peptides and polypeptides encoded thereby. They can be used inter alia to facilitate therapeutic replacement of polypeptide variants encoded by genetic polymorphisms, particularly such polymorphisms associated with inherited disease. The capped, circularized RNA molecules provided by the invention advantageously can provide transient expression of an encoded peptide or polypeptide, thus providing therapeutic flexibilities conventional gene replacement therapies have been unable to achieve. The capped, circularized RNA molecules provided by the invention possess the advantages that other circular RNA process, including resistance to exonucleases and higher ribosome loading. The capped, circularized RNA molecules provided by the invention further advantageously provide translation initiation not limited to internal ribosome entry sites (IRES) or translation enhancing elements (TEE), providing more robust cap-dependent translation initiation thereby. Another advantage of the capped, circularized RNA molecules provided by the invention is to replace protein-based therapeutics (i.e., wherein the protein is delivered and must be introduced specifically into target cells in functional fashion and be targeted to the proper intracellular niche; see, Lagasse et al., 2017, F1000 Research 6: 113; doi:10.12688/f1000research.9970.1) with delivery of RNA encoding the necessary peptide or polypeptide in a form (capped, circular) that is resistant to exonuclease degradation and provided robust expression due to the presence of eukaryotic cap.

These and other features, objects, and advantages of the invention will become better understood from the description that follows. In the description, reference is made to the accompanying drawings, which form a part hereof and in which there is shown by way of illustration, not limitation, embodiments of the invention. The description of preferred embodiments is not intended to limit the invention to cover all modifications, equivalents, and alternatives. Reference should therefore be made to the claims recited herein for interpreting the scope of the invention.

### BRIEF DESCRIPTION OF THE DRAWINGS

FIGS. 1A-1H show conceptualization of capped circular mRNA (QRNA). FIG. 1A shows schematics of regular circular RNA structure and IRES mediated translation initiation. FIG. 1B shows schematics of regular linear RNA structure and m7G-cap dependent translation initiation. FIG. 1C illustrates design of a capped-circular mRNA (QRNA): a circular RNA bearing a click reaction handle in its 5′ untranslated region (UTR) is chemically conjugated to the 3′-end of a linear oligo, which is chemically capped. QRNA hijacks the cap-dependent translation initiation mechanism to increase ribosome loading on the circular transcript. FIG. 1D and FIG. 1E show generic structures of type 1&2 QRNAs. FIG. 1F illustrates the synthetic scheme of a Type 3 Flag-encoding QRNA, wherein Oligo 3.1 is a sequence-designed RNA containing twister ribozymes (red), tRNA-like hairpin (green), 5′UTR (blue), and 3×Flag-peptide coding sequences (orange); Oligo 3.2 is a circularized RNA; Oligo 3.3 is a circular RNA bearing a 5-methyl-tetrazine click chemistry handle; Oligo 3.4 is a capped oligo with a 3′-TCO click chemistry handle; and Oligo 3.5: is the desired 3×Flag encoding Type 2 QRNA product with click chemistry linkage shown in pink. FIG. 1G illustrates the structure of 5-methyl-tetrazine containing preQ1 cofactor analogue. FIG. 1H shows the structure of the 3′-TCO click chemistry handle.

FIGS. 2A-2H show a general synthesis workflow of Type 1 QRNA. FIG. 2A shows representative chemical structures of various components in the capped circular mRNA: cap, alkyne handles, phosphate group, and azide handles, each is color coded and shown in the following figures. FIG. 2B illustrates a chemically synthesized oligo bearing a 3′-end click chemistry handle is chemically capped and high-performance liquid chromatography (HPLC) purified. FIG. 2C shows alternatively a chemically synthesized oligo bearing a 3′-end click chemistry handle can be enzymatically capped. FIG. 2D illustrates a chemically/enzymatically synthesized mRNA transcript bearing a click chemistry handle in its 5′-UTR and a 5′-phosphate can be circularized using T4 RNA ligase; complementary sequences in both 5′ and 3′ UTRs facilitate the circularization. CircRNA bearing the click chemistry handle is chemically conjugated to the capped oligo. FIG. 2E shows a chemically/enzymatically synthesized mRNA transcript bearing a click chemistry handle in its 5′-UTR and a 5′-phosphate can be circularized using T4 RNA ligase II using a DNA splint probe complementary to the 5′/3′-ends. CircRNA bearing the click chemistry handle is chemically conjugated to the capped oligo. FIG. 2F shows mRNA constructs bearing a RNA stem-loop in its 5′-UTR can be synthesized by IVT and circularized using methods in 2C/2D, or circularized using ribozyme mediated backsplicing. The stem-loop can be modified using RNA-modifying enzymes such as tRNA transferases to introduce a click reaction handle on the circular RNA. CircRNA bearing the click chemistry handle is chemically conjugated to the capped oligo. FIG. 2G demonstrates in vitro transcribed mRNA having 3′/5′-hydroxyl groups can be enzymatically ligated to a chemically synthesized oligo bearing a click chemistry handle and phosphates on both 5′ and 3′ ends. Ligation of 5′-phosphate and 3′-OH is achieved using T4 RNA ligase II and ligation of 5′-OH and 3′-phosphate is achieved using RNA ligase RtcB. Both ligation is facilitated by DNA splint. CircRNA bearing the click chemistry handle is chemically conjugated to the capped oligo. FIG. 2H demonstrates in vitro transcribed mRNA 5′ triphosphate/3′ hydroxyl groups can be enzymatically ligated to a chemically synthesized oligo bearing a click chemistry handle and phosphates on both 5′ and 3′ ends. Ligation of 5′-phosphate and 3′-OH is achieved using T4 RNA ligase II. 5′-triphosphate is hydrolyzed using calf intestinal alkaline phosphatase (CIAP) to a 5′-hydroxyl. Ligation of 5′-OH and 3′-phosphate is achieved using RNA ligase RtcB. Both ligation is facilitated by DNA splint. CircRNA bearing the click chemistry handle is chemically conjugated to the capped oligo. FIG. 2I shows alternatively a chemically synthesized oligo bearing a click chemistry handle can be used as primers annealing to the DNA template and in vitro transcribed using an RNA polymerase engineered from DNA polymerase. The IVT-synthesized mRNA can be then used for QRNA synthesis as outlined in FIG. 2D/FIG. 2E.

FIGS. 3A-3B demonstrates a general synthesis workflow of Type 2 QRNA. FIG. 3A shows that a capped RNA having a stem-loop in its 5′-UTR is synthesized by IVT and subsequently ligated to chemically synthesized oligos having 5′-phosphate and 3′-click chemistry handles using T4 RNA ligase. The product is intramolecularly circularized to yield type 2 QRNA. FIG. 3B shows alternatively a chemically synthesized oligo bearing a click chemistry handle is capped and used as primers annealing to the DNA template and in vitro transcribed using an RNA polymerase engineered from DNA polymerase. The IVT-synthesized mRNA can be then used for type 2 QRNA synthesis.

FIGS. 4A-4F show a proof-of-concept experiment using Type 1 QRNA encoding HiBit tag. FIG. 4A shows synthesis of type 1 HiBit QRNA. HiBit-encoding RNA was codon optimized such that it only contains one single C in the 5′-UTR and an azide handle was incorporated by full replacement of CTP with azide CTP during IVT. FIG. 4B show representative HPLC traces and gel electrophoresis of azide-circRNA purification. FIG. 4C is a representative HPLC trace of chemically-capped EU-containing oligo. FIG. 4D shows precursors 1-6 produced during the synthesis reaction. FIG. 4E is gel electrophoresis characterization of Hibit QRNA. FIG. 4F show barplots of HiBit QRNAs luminescence 8 hrs upon transfection in HeLa cell. Mean±sem. P values were calculated by unpaired t test with Welch's correction. ****P<0.0001; **P<0.0021; *P<0.0332. n.s. P>0.1234.

FIGS. 5A-5B show a summary of oligonucleotide chemical conjugation methods. Screening was performed using 15-nt dA model substrates at micromolar concentrations. Modification handles were incorporated through solid phase synthesis, followed by amine-NHS labeling and HPLC purification if necessary. FIG. 5C is gel electrophoresis of crude thiol-ene/yne oligonucleotide conjugation of 15-nt model substrates containing only one conjugation handle. FIG. 5D is gel electrophoresis of crude CuAAC and IEDDA 30-nt oligonucleotides bearing three EU/TCO handles reacting with 30-nt N₃/Tz modified oligo.

FIG. 6 shows chemical structures of engineered branched poly(A) tails. Branching oligos are conjugated through triazole linkage and flanked by 15 A's in between. Nuclease-resistant modifications are incorporated on the last 6 nucleotides of each branch. Chain terminating nucleotides are introduced at 3′-end to prevent self-ligation.

FIGS. 7A-7F shows synthesis of 100% capped, chemically and topologically augmented oligonucleotides. FIG. 7A shows an illustration of chemical capping and HPLC purification of solid-phase synthesized oligonucleotides and graphs of capped oligos produced using methods disclosed here. FIG. 7B shows scalability of oligo capping in ranges between 4-12 nanomoles. FIG. 7C shows PAGE characterization of uncapped/fully-capped oligonucleotides (15% TBU). FIG. 7D are graphs of HPLC purification of capped oligos with various sugar backbones. FIG. 7E is a graph showing HPLC purification of branched oligo with two caps. FIG. 7F shows PAGE characterization of dual-capped oligo (15% TBU) having the structures shown graphically. M, marker.

FIGS. 8A-8H shows multidimensional chemical optimization of the mRNA cap and 5′-UTR. FIG. 8A is a graphical illustration showing accessing the 5′-mRNA chemical modification landscape using an integrated chemo-enzymatic approach. FIGS. 8B-8E shows barplots of time-course dual luciferase assay screening chemical modifications on first base identity, phosphodiester linkage, sugar backbone, and m⁷G cap as illustrated for each bar graph corresponding thereto in the Figures. Protein expression was measured by Firefly luciferase luminescence normalized to Renilla luciferase luminescence (transfection control) then to the wild-type cap (m⁷G-G) construct (red line) at 8, 24, 48 hrs post transfection. Mean±sem. P values were calculated by ordinary one-way ANOVA (alpha=0.05), with multiple comparisons to m⁷G-G (panel b)/m⁷G-A (FIG. 8C-8E) at 24 hr. *P<0.0332, **P<0.0021, ***P<0.0002, ****P<0.0001, n.s. P>0.05. FIG. 8F shows combinatorial optimization of cap and 5′-UTR sugar backbone modifications. FIG. 8G is a graph showing effects of chemical modifications on oligonucleotide affinity towards eIF4E by EMSA. FIG. 8H is a graph showing effects of chemical modifications on oligonucleotide resistance against hDcp2.

FIGS. 9A-9B show chemo-topological engineering of multi-capped branched mRNA. FIG. 9A is a comparison of a branched-cap with a regular cap with bioluminometry (8 hrs post transfection). FIG. 9B are bar graphs showing bioluminescence decay of a branch cap versus regular cap from 8 hrs to 24 hrs post transfection. P values were calculated by unpaired t test. ***P<0.001; ****P<0.0001; n.s. P>0.05.

FIGS. 10A-10G show synthesis of capped-circular mRNA via branched topology. FIG. 10A shows IRES-based circRNA translation initiation through eIF4G-4F complex. FIG. 10B shows branched cap induced translation while lacking protection of mRNA from exonuclease degradation. FIG. 10C shows conceptualization of capped-circular mRNA (QRNA) to snatch eIF4E dependent pathways for circRNA translation initiation. FIG. 10D is an illustration of QRNA synthesis workflow by combining enzymatic labeling and click chemistry. FIG. 10E is an illustration of double RNase H assay characterization of QRNA and PAGE analysis thereof. FIG. 10F shows QRNA effectively induced translation on circRNA without IRES. m⁷G capped, linear mRNA with wild-type uridine served as a positive control. circRNA bearing an iHRV IRES was also compared. Translation activities were measured 6 hours post transfection. FIG. 10G shows that uridine reduces mRNA translation globally. m⁷G capped linear mRNA with/without m¹Ψ and QRNA (with uridine) encoding Nluc were co-transfected with Flue mRNA (m⁷G capped, m¹Ψ modified). Translations of controlled Flue mRNA were also reduced when uridine containing Nluc mRNA was transfected.

### DETAILED DESCRIPTION

Provided herewith is a more detailed description of the compositions, reagents, and methods comprising the invention, which is provided to explain and enhance but not replace or be a substitute for the claims set forth below.

All publications, including but not limited to patents and patent applications, cited in this specification are herein incorporated by reference as though set forth in their entirety in this application.

### Definitions

As used in the specification, articles “a” and “an” are used herein to refer to one or to more than one (i.e., at least one) of the grammatical object of the article. By way of example, “an element” means at least one element and can include more than one element.

“About” is used to provide flexibility to a numerical range endpoint by providing that a given value can be “slightly above” or “slightly below” the endpoint without affecting the desired result. The term “about” in association with a numerical value means that the numerical value can vary by plus or minus 5% or less of the numerical value.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements, or steps but not the exclusion of any other integer or step or group of integers or steps.

As used herein, “and/or” refers to and encompasses any and all possible combinations of one or more of the associated listed items, as well as the lack of combinations where interpreted in the alternative (“or”).

Recitation of ranges of values herein are merely intended to serve as a succinct method of referring individually to each separate value falling within the range, unless otherwise indicated herein. Furthermore, each separate value is incorporated into the specification as if it were individually recited herein. For example, if a range is stated as 1 to 50, it is intended that values such as 2 to 4, 10 to 30, or 1 to 3, etc., are expressly enumerated in this disclosure. These are only examples of what is specifically intended, and all possible combinations of numerical values between and including the lowest value and the highest value enumerated are to be considered to be expressly stated in this disclosure.

As utilized in accordance with the present disclosure, unless otherwise indicated, all technical and scientific terms shall be understood to have the same meaning as commonly understood by one of ordinary skill in the art. Unless otherwise required by context, singular terms shall include pluralities and plural terms shall include the singular.

This application discloses and claims compositions, reagents, and methods comprising capped, circular RNA molecules, particularly circularized mRNA molecules preferably encoding a peptide or polypeptide. As provided herein, the cap used in the capped, circularized RNA molecules of the invention can include 7-methylguanine (m⁷G) but in addition cap analogues as set forth, inter alia, in U.S. patent application No. 2020/0055891 to Walczak et al.; Holstein et al., 2016, Agnew Chem. Int. Ed. Engl. 55: 10899-10903; Walczak et al., 2017, Chem. Sci. 8: 260-267; Muttach et al., 2017, J. Org. Chem. 13: 2819-2832) can be incorporated into the circular RNA molecule precursors to create the capped, circularized RNA molecules provided herein.

Capped-Circular mRNA (QRNA)

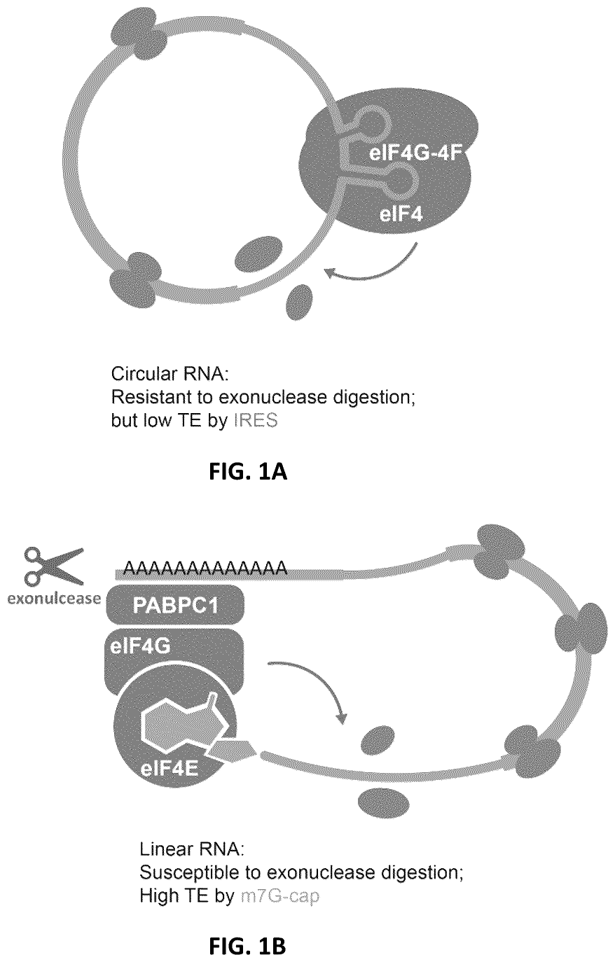

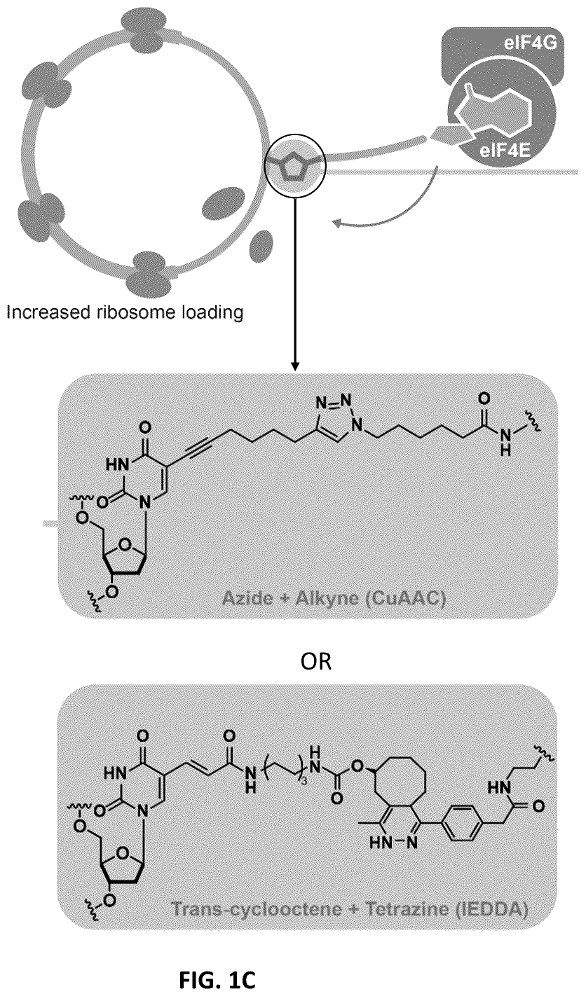

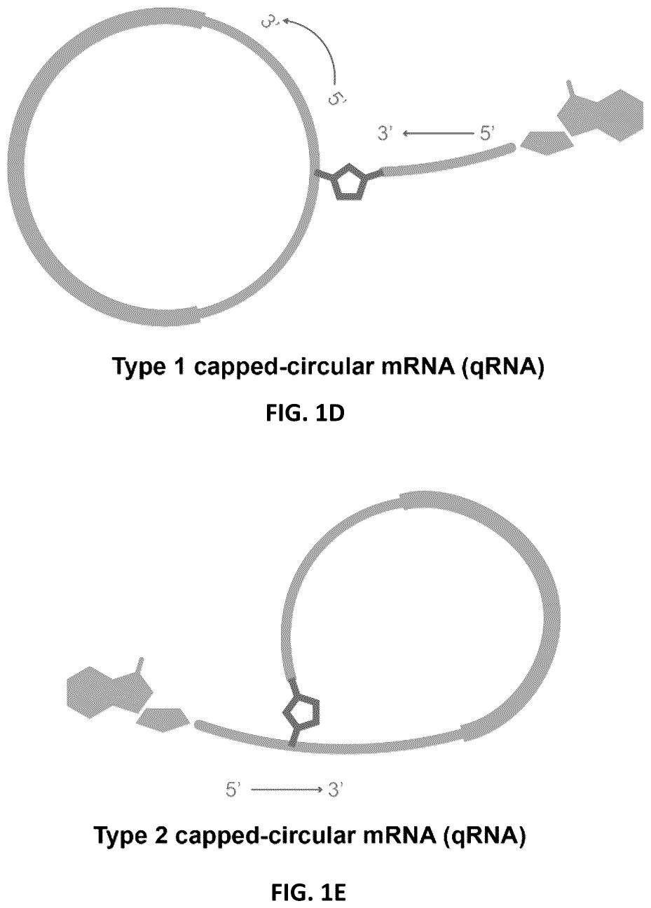

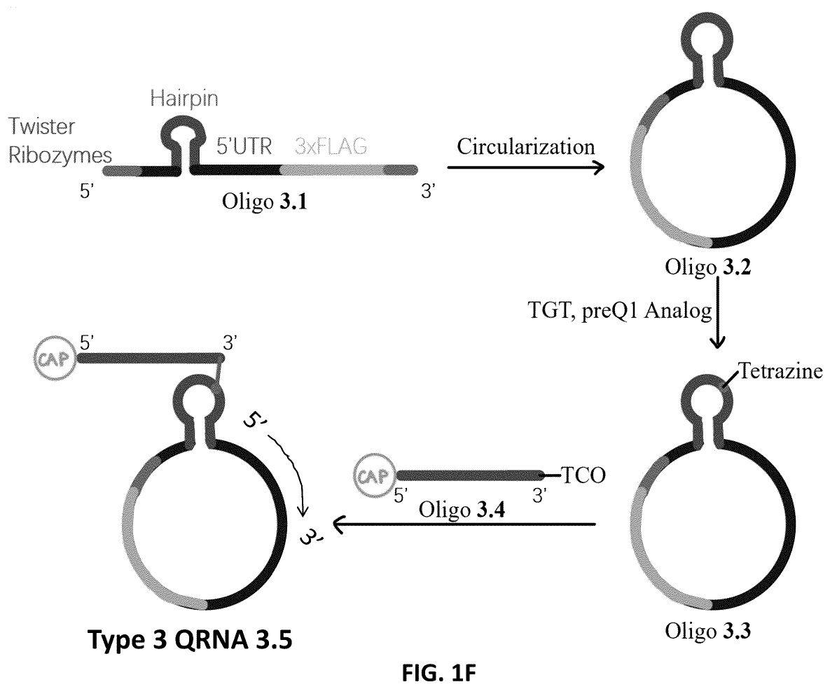

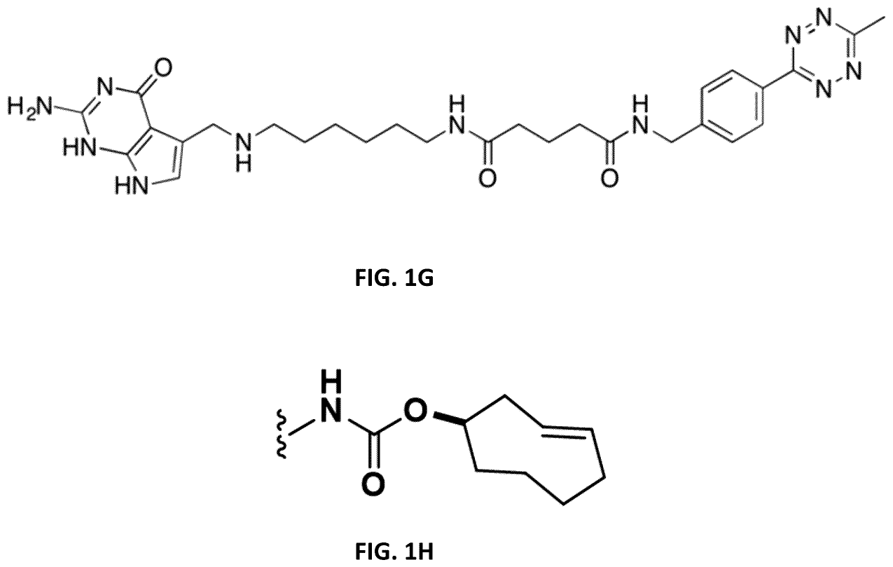

Despite advances in circular RNA (circRNA) engineering, current constructs rely on IRES (Internal Ribosome Entry Site) or TEE (Translation Enhancing Element)-mediated translation, which are embodiments that enable cap-independent translation (FIG. 1A). Linear mRNAs are capable of undergoing cap-dependent translation through interaction with eIF4E and other eukaryotic translation initiation factors (FIG. 1B), which is the predominant form of translation in cells (Sonenberg and Hinnebusch, 2009, Cell 136: 731-745) and is generally more efficient than cap-independent translation (Koch et al., 2020, Nat. Struct. Mol. Biol. 27:1095-1104).

As described herein, a “capped-circular mRNA” is a circular mRNA characterized by one or more covalent linkages to one or more cap structures (or a derivative thereof). The circular mRNA can contain all the canonical elements of a linear mRNA: (1) Cap, (2) 5′ UTR (untranslated region), (3) protein-coding regions (CDS), (4) 3′ UTR, and (5) poly(A) tail. By circularizing these features into a capped-circular RNA, it is intended to enhance half-life (increased nuclease resistance) of a canonical circular mRNA, while retaining the benefits of efficient cap-dependent translation, such as in linear mRNA.

The RNA embodiments and methods disclosed herein take advantage of the exonuclease-resistant feature of circRNA while utilizing the strong m7G-cap dependent translation initiation machinery. Such features can be achieved via chemical conjugation of a capped oligonucleotide with a circRNA through click chemistries such as copper catalyzed azide-alkyne cycloaddition (CuAAC) or tetrazine-trans cyclooctene inverse electron demand Diels-Alder reaction (IEDDA) (FIG. 1C). As shown in FIG. 1D and FIG. 1E, the invention contemplates two generic structures of capped circular messenger RNAs (QRNAs): Type 1 QRNA and Type 2 QRNA. In Type 1 QRNA, a circular poly-phosphodiester backbone is present while capping is achieved via chemical ligation of a short, capped oligonucleotide to an internal handle on the circular mRNA through click chemistry. The 5′ cap may comprise of a 7-methylguanylate that enables efficient translation of an mRNA or alternative common mRNA cap structures, as shown, for example, in Mccaffreyanton, 2019, Genetic Engineering & Biotechnology News. 39. In Type 2 QRNA, a continuous mRNA poly-phosphodiester backbone is present; circularization is achieved via chemical conjugation between the 3′-end and 5′-UTR of the mRNA through click chemistry.

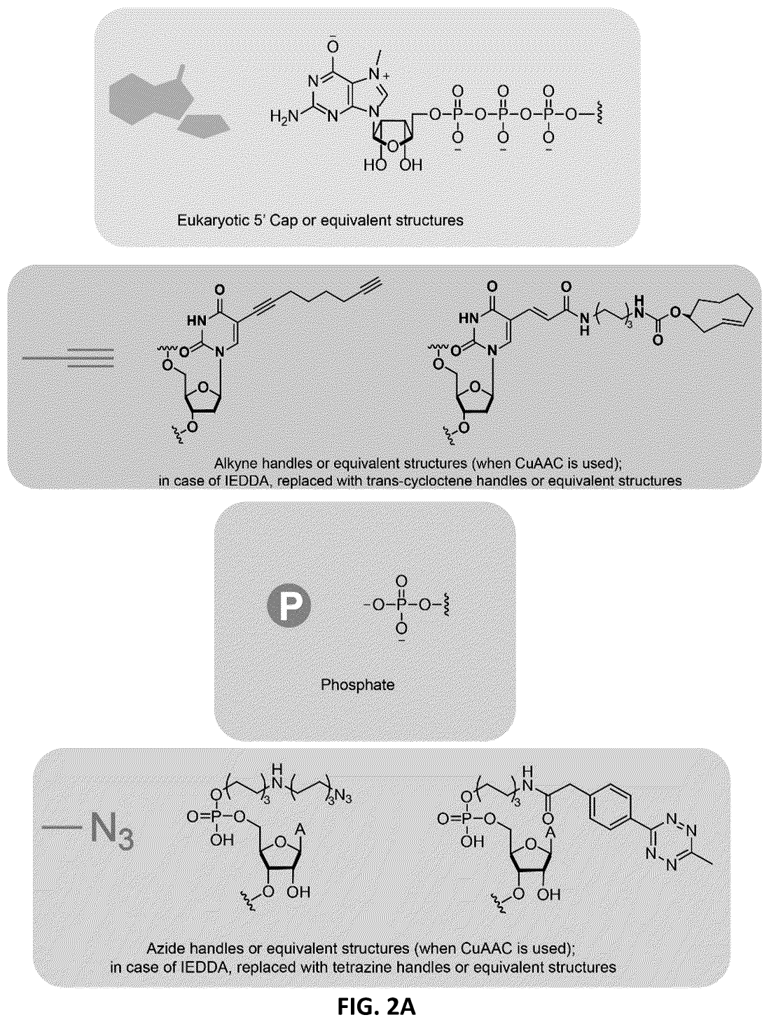

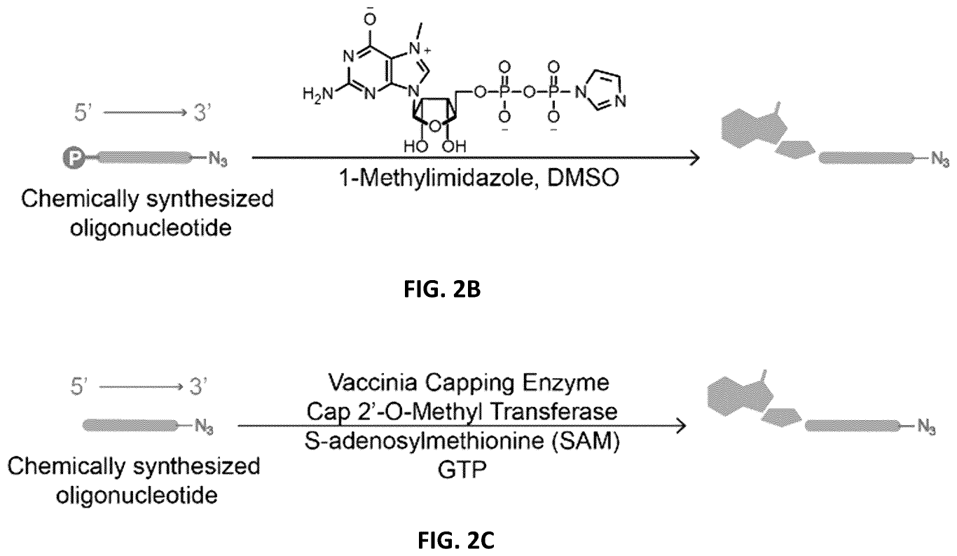

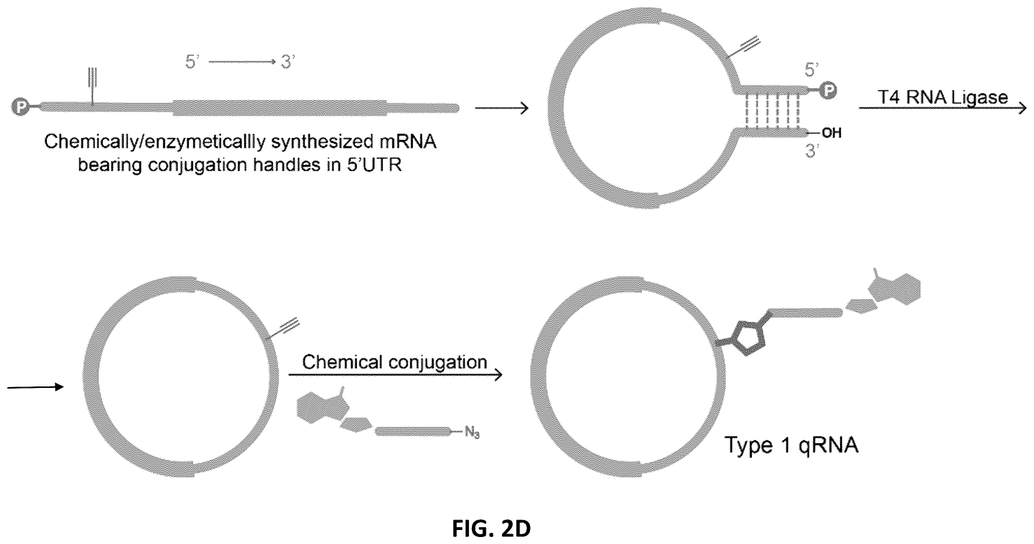

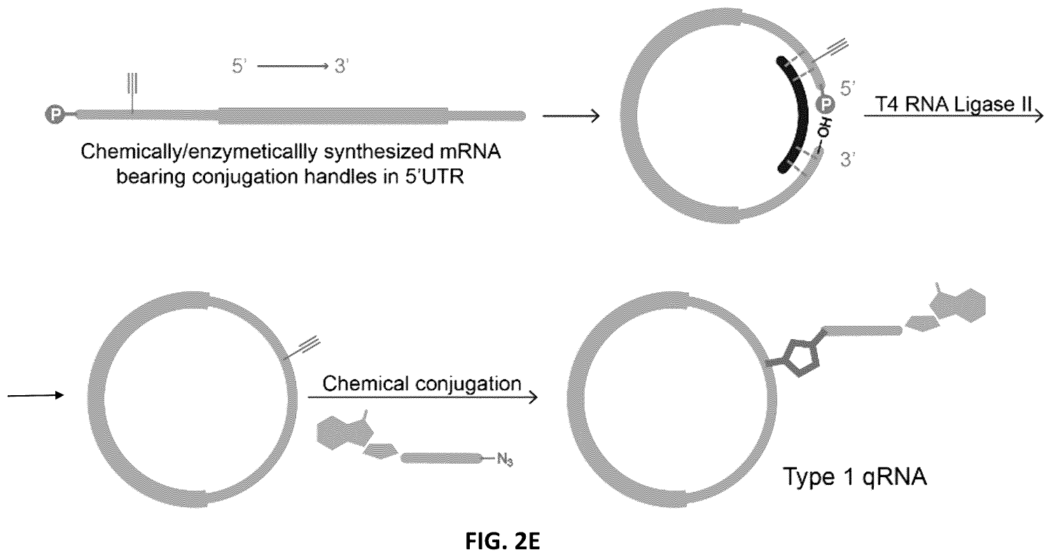

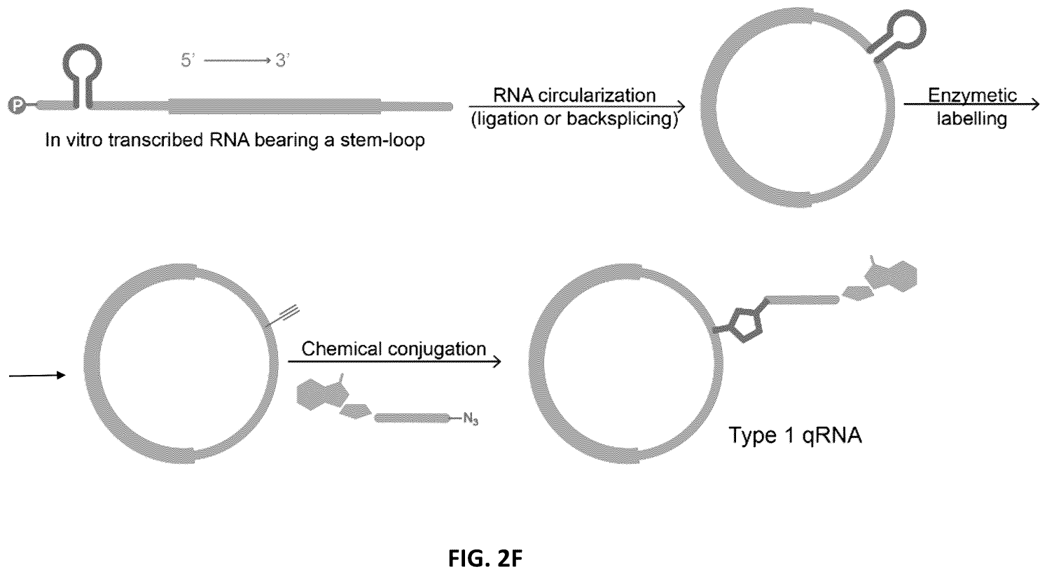

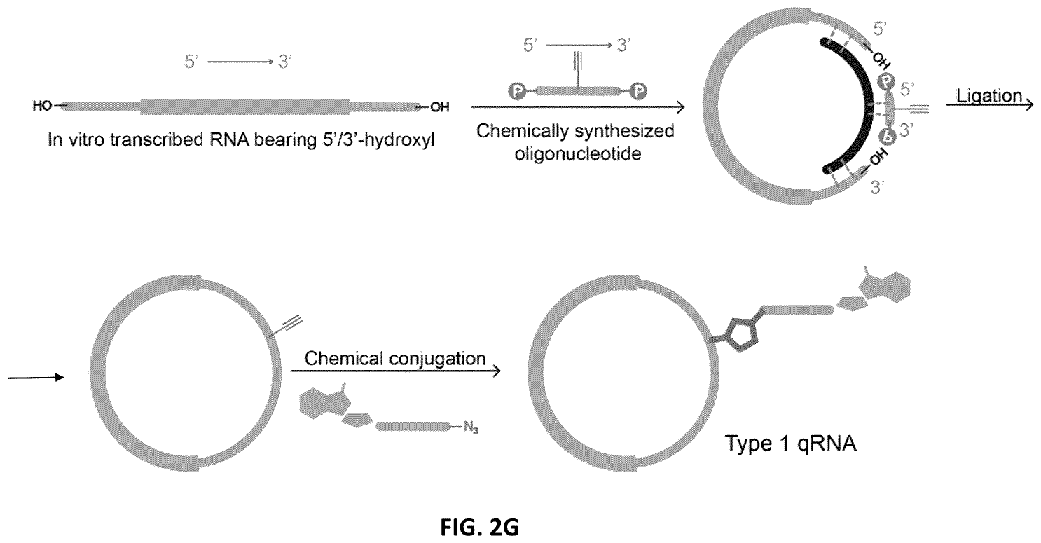

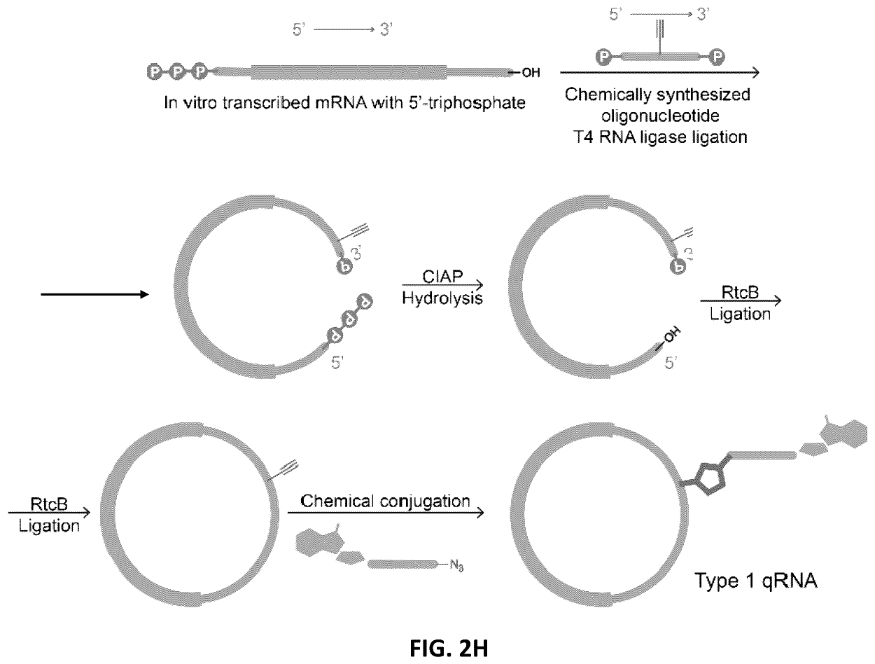

Various components of the circRNA are depicted in FIG. 2A. The 5′ capping and 3′ poly(A) tailing steps are useful in producing active synthetic mRNA; these modifications prevent mRNA degradation and facilitate translation initiation in eukaryotic cells. As used herein, “capping” means modification at the 5′ end of an mRNA by an addition of a “cap” molecule such as a 7-methylguanosine (m⁷G) cap. Other cap structures and modifications of the cap as described below can be used to optimize the translation efficiency.

Enzymes capable of catalyzing the reaction of linking a cap molecule to the mRNA include, but are not limited to, Vaccinia capping system including 2′-O-Methyl Transferase (FIG. 2C), tRNA guanine transglycosylase (TGT), Faustovirus capping enzyme, and T4-RNA ligase. Capping can also occur during the synthesis of mRNA called co-transcriptional capping.

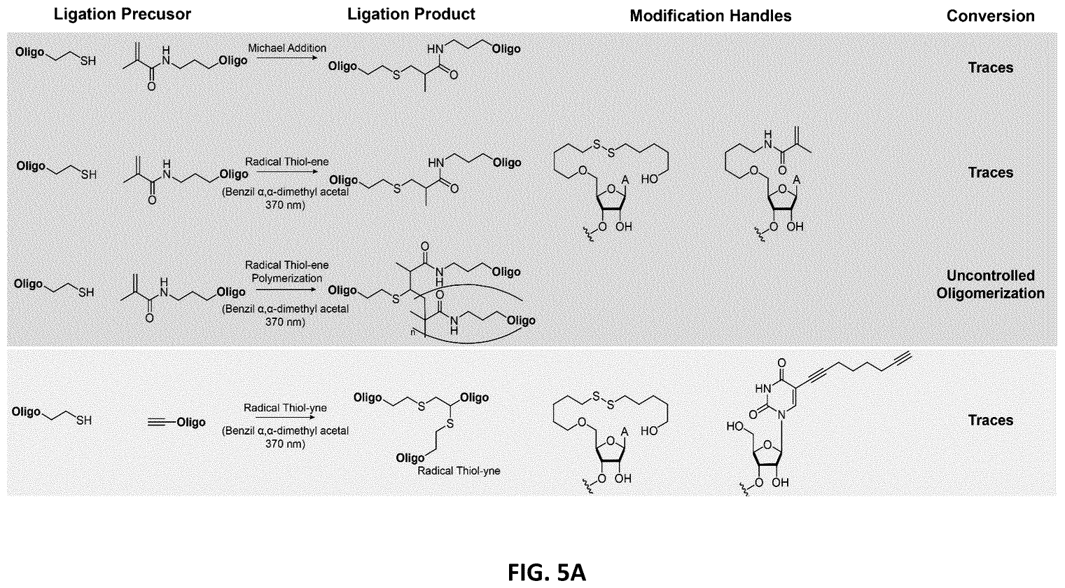

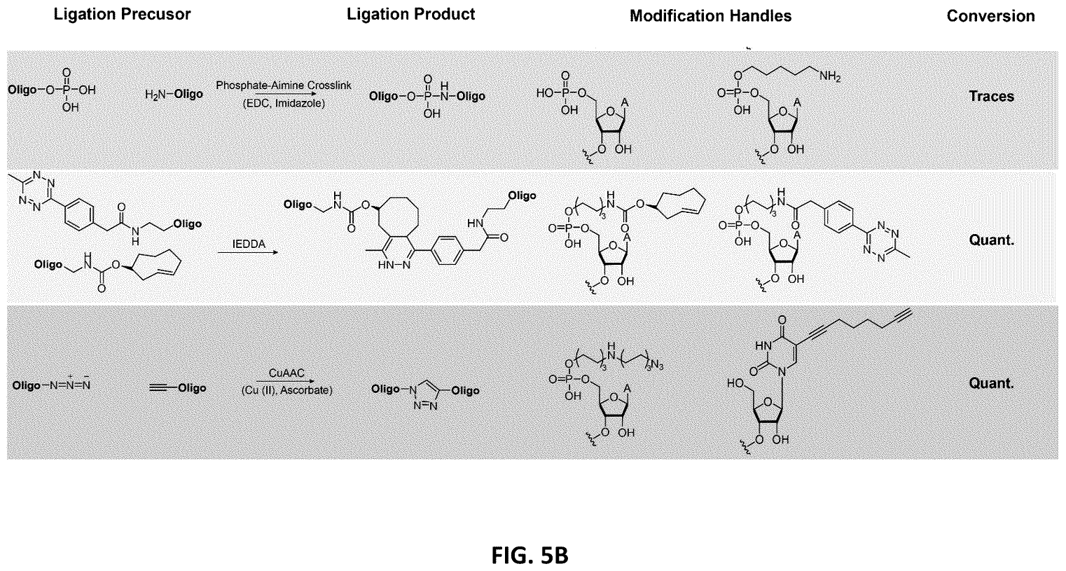

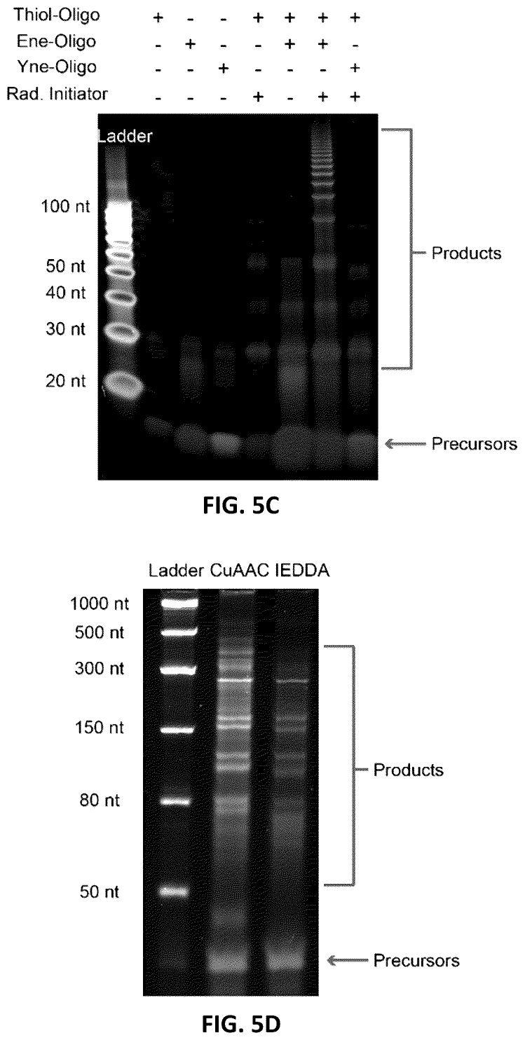

As used herein, the term “molecular handle” or “handle” refers to a chemical group that is attached to a nucleotide on mRNA and can form a covalent bond to another molecule that is separate from the mRNA to link this other molecule to the mRNA. The covalent bond can be formed via various appropriate functional crosslinking reactions. In some embodiments described herein, the crosslinking reaction is click chemistry. As used herein, the term “click handle” refers to a molecule on mRNA that can covalently bind to another molecule via click chemistry reaction. Examples of a handle include, but are not limited to, alkyne or azide (when CuAAC is used in click chemistry), or trans-cyclotene or tetrazine (when IEDDA is used in click chemistry), or hydrozone or oxime, or any equivalent structures thereof. Other crosslinking chemistries including thio-ene and tiol-yne reactions (Escorihuela et al., 2014, Bioconjug. Chem. 25:618-627), a phosphate-amine based reaction (El-Sagheer and Brown, 2017, Chem. Commun. 53:10700-10702; Kalinowski et al., 2016, Chembiochem. 17: 1150-1155) (shown in FIG. 5A and FIG. 5B respectively), thiol-yne, amino-yne, and hydroxyl-yne reactions (Worch et al., 2021, Chem Rev. 121(12): 6744-6776), and other bioconjugation reactions (Gassensmith, https://chem.libretexts.org/Bookshelves/Organic_Chemistry/Supplemental_Modules_(Organic_Chemistry)/Reactions/Introduction_to_Bioconjugation, accessed Jun. 23, 2023) have also been contemplated.

As used herein, the term “hairpin” or “hairpin oligonucleotide” refers to a single-stranded oligonucleotide that has a sequence of complementary base pairs at both ends capable of forming a “stem-and-loop” structure.

As used herein and understood in the art, the term “click chemistry” is intended to encompass chemical methods for linking chemical components together, including but not limited to nucleotides into polynucleotides and amino acids into peptides and polypeptides, that are “simple to perform, have high yields, require no or minimal purification, and are versatile in joining diverse structures without the prerequisite of protection steps” (see, for example, Hein et al., 2006, Pharm. Res. 10: 2216-2230). In current chemical synthetic practice four primary reactions are employed: 1) cycloadditions (including for example monovalent copper-catalyzed Huisgen 1,3-dipolar cycloadditions of azides and alkynes, the most widely used); 2) nucleophilic ring openings (including ring systems comprising strained heterocyclic electrophiles); 3) non-Aldol carbonyl chemistry (including for example hydrazone/oxime ether formation); and 4) carbon multiple bond additions (including for example certain Michael additions and formation of various three-membered rings by inter alia epoxidations). Click chemistry has been found to be particularly useful for polymeric substances such as proteins and nucleic acids as illustrated herein.

As used herein, the term “equivalent structure” means any molecule that are sufficiently structurally similar and perform the same function in a chemical reaction.

As used herein, the terms “derivatized” or “functionalized” means modification of a nucleotide that leads to some functional consequences in its chemical properties or reactivity or both. Both terms shall be understood to be equivalent to the extent that particular embodiments of the capped, circular RNA molecules have by benefit of derivatization thereof a function, particularly with regard to crosslink-dependent circularization embodiments provided herein. In some embodiments, a derivatized nucleotide is a nucleotide that is modified to comprise a chemical group/handle can participate in a cross-linking reaction.

As used herein, the term “QRNA” is intended as a generic term meaning capped circular messenger RNAs. Particularly encompassed by this term are the various species of circularized RNA molecules and in particular circularized mRNA molecules disclosed herein, but these examples are not intended to be limiting.

In some embodiments, the synthesis pathway of Type 1 and Type 3 QRNA enables multiple oligonucleotides containing 5′ cap binding to the circular RNA. For example, circular RNA can include multiple derivatized nucleotides that can covalently bind to multiple oligonucleotides containing 5′ cap. Alternatively, a single circular RNA backbone can encode multiple TGT sites to enable binding of multiple oligonucleotides containing 5′ cap onto the circular RNA simultaneously.

In some embodiments, the capped, circular RNA molecule comprises an mRNA region encoding one or a plurality of peptides or polypeptides.

### Cap Modifications

Several variations of the cap structure have been contemplated here to optimize translation efficiency of QRNA. These variations include: including multiple cap structures (cap 0, 1, and 2; Shanmugasundaram et al., 2022, Chem Rec. 22(8): e202200005); including N⁶, 2′-O-dimethyladenosine (m⁶Am) as a terminal modification adjacent to the mRNA cap (Sun et al., 2021, Nat Commun. 12(1): 4778); using cap structures with modified triphosphate bridges (Sun et al., 2021, Nat Commun. 12(1): 4778; Wojtczak et al., 2018, J Am Chem Soc. 140(18): 5987-5999); incorporating Locked Nucleic Acid (LNA)-modified cap analogs (Kore et al., 2009, J Am Chem Soc. 131(18): 6364-5); introducing cap analogs with alternative functionalities such as light reactivity and click groups (Klocker et al., 2022, Nat Chem. 14(8): 905-913; Nowakowska et al., 2014, Org. biomol. Chem. 12: 4841-4847); hydrophobic cap analogs (WO 2017066782 A1); and others (Wojcik et al., 2021, Pharmaceutics 13(11): 1941; Grudzien et al., RNA 10(9): 1479-1487; Grzela et al., 2023, RNA 29(2): 200-216).

In some embodiments, the methyl group in 7-methylguanosine (m⁷G) cap structure can be modified to produce 7-benzylguanosine (Bn⁷G), 7-chlorobenzylguanosine (ClBn⁷G), and chlorobenzyl-O-ethoxyguanosine (ClBnOEt⁷G). Introduction of one or more Locked Nucleic Acid (LNA), 2′-methoxy (2OMe), and 2-methoxyethoxy (2MOE) into m⁷G structure significantly increase mRNA translation. In some embodiments, the cap structures include, but are not limited to, m⁷G-LNA, LNAm⁷G-LNA, LNAm⁷G-LNAx6, LNAm⁷G-2OMex6. In some embodiments, the cap structure is m⁷G diphosphate imidazolide (m⁷GDP-Im).

### Nucleotide Modifications

In some embodiments, as disclosed and recognized herein it is beneficial to alter the type of nucleotide/nucleotide identity, specifically incorporation of adenosine (A), guanosine (G), 6-methyladenosine (m⁶A), or the non-canonical inosine (I) in the mRNA, preferably, at the +1 position, increases translation efficiency. In some embodiments, substitution of some or all uridine residues to N′-methylpseudouridine (m¹Ψ) in the mRNA also boosts the translation. The nucleotides are numbered according to their position immediately downstream of the cap structure. For example, the cap structure found at the 5′ end of eukaryotic mRNAs consists of a 7-methylguanosine (m⁷G) moiety linked to the first nucleotide (+1 position) of the transcript via a 5′-5′ triphosphate bridge.

Other modified nucleotides include, but are not limited to, pseudouridine, 5-methylcytidine, 2-thiouridine, 5-methoxyuridine, 4-acetylcytidine, xanthine, allyaminouracil, allyaminothymidine, hypoxanthine, digoxigeninated adenine, digoxigeninated cytosine, digoxigeninated guanine, digoxigeninated uracil, 6-chloropurineriboside, N6-methyladenine, methylpseudouracil, 2-thiocytosine, 2-thiouracil, 5-methyluracil, 4-thiothymidine, 4-thiouracil, 5,6-dihydro-5-methyluracil, 5,6-dihydrouracil, 5-[(3-Indolyl)propionamide-N-allyl]uracil, 5-aminoallylcytosine, 5-aminoallyluracil, 5-bromouracil, 5-bromocytosine, 5-carboxycytosine, 5-carboxymethylesteruracil, 5-carboxyuracil, 5-fluorouracil, 5-formylcytosine, 5-formyluracil, 5-hydroxycytosine, 5-hydroxymethylcytosine, 5-hydroxymethyluracil, 5-hydroxyuracil, 5-iodocytosine, 5-iodouracil, 5-methoxycytosine, 5-methoxyuracil, 5-methylcytosine, 5-methyluracil, 5-propargylaminocytosine, 5-propargylaminouracil, 5-propynylcytosine, 5-propynyluracil, 6-azacytosine, 6-azauracil, 6-chloropurine, 6-thioguanine, 7-deazaadenine, 7-deazaguanine, 7-deaza-7-propargylaminoadenine, 7-deaza-7-propargylaminoguanine, 8-azaadenine, 8-azidoadenine, 8-chloroadenine, 8-oxoadenine, 8-oxoguanine, araadenine, aracytosine, araguanine, arauracil, biotin-16-7-deaza-7-propargylaminoguanine, biotin-16-aminoallylcytosine, biotin-16-aminoallyluracil, cyanine 3-5-propargylaminocytosine, cyanine 3-6-propargylaminouracil, cyanine 3-aminoallylcytosine, cyanine 3-aminoallyluracil, cyanine 5-6-propargylaminocytosine, cyanine 5-6-propargylaminouracil, cyanine 5-aminoallylcytosine, cyanine 5-aminoallyluracil, cyanine 7-aminoallyluracil, dabcyl-5-3-aminoallyluracil, desthiobiotin-16-aminoallyl-uracil, desthiobiotin-6-aminoallylcytosine, isoguanine, N1-ethylpseudouracil, N1-methoxymethylpseudouracil, N1-methyladenine, N1-methylpseudouracil, N1-propylpseudouracil, N2-methylguanine, N4-biotin-OBEA-cytosine, N4-methylcytosine, N6-methyladenine, O6-methylguanine, pseudoisocytosine, pseudouracil, thienocytosine, thienoguanine, thienouracil, xanthosine, 3-deazaadenine, 2,6-diaminoadenine, 2,6-daminoguanine, 5-carboxamide-uracil, 5-ethynyluracil, N6-isopentenyladenine (i6A), 2-methyl-thio-N6-isopentenyladenine (ms2i6A), 2-methylthio-N6-methyladenine (ms2m6A), N6-(cis-hydroxyisopentenyl)adenine (io6A), 2-methylthio-N6-(cis-hydroxyisopentenyl)adenine (ms2io6A), N6-glycinylcarbamoyladenine (g6A), N6-threonylcarbamoyladenine (t6A), 2-methylthio-N6-threonyl carbamoyladenine (ms2t6A), N6-methyl-N6-threonylcarbamoyladenine (m6t6A), N6-hydroxynorvalylcarbamoyladenine (hn6A), 2-methylthio-N6-hydroxynorvalyl carbamoyladenine (ms2hn6A), N6,N6-dimethyladenine (m62A), and N6-acetyladenine (ac6A) have also been contemplated at +1 and other positions.

In some embodiments, the modified phosphate backbone can be phosphorothioate (PS), thiophosphate, 5′-O-methylphosphonate, 3′-O-methylphosphonate, 5-hydroxyphosphonate, hydroxyphosphanate, phosphoroselenoate, selenophosphate, phosphoramidate, carbophosphonate, methylphosphonate, phenylphosphonate, ethylphosphonate, H-phosphonate, guanidinium ring, triazole ring, boranophosphate (BP), methylphosphonate, or guanidinopropyl phosphoramidate.

In some embodiments, introduction of locked nucleic acid (LNA), 2′-methoxyribose (2-OMe), and 2-methoxyethoxy (2-MOE) into the ribose sugar backbone increases mRNA translation. Addition of multiple 2-OMe and 2-MOE modified bases increases translation further. LNA specifically increased expression at the +1 position.

In some embodiments, the modified sugar can be 2-thioribose, 2,3-dideoxyribose, 2-amino-2-deoxyribose, 2′ deoxyribose, 2′-azido-2′-deoxyribose, 2′-fluoro-2′-deoxyribose, 2′-O-methylribose, 2′-O-methyldeoxyribose, 3′-amino-2′, 3′-dideoxyribose, 3′-azido-2,3-dideoxyribose, 3′-deoxyribose, 3′-O-(2-nitrobenzyl)-2′-deoxyribose, 3′-O-methylribose, 5′-aminoribose, 5′-thioribose, 5-nitro-1-indolyl-2′-deoxyribose, 5′-biotin-ribose, 2′-0,4′-C-methylene-linked, 2′-0,4′-C-amino-linked ribose, or 2′-0,4′-C-thio-linked ribose.

In these backbone modifications, stereoisomer structures are also considered since they have been shown to impact the RNA's nuclease-resistance properties (Iwamoto et al., 2017, Nat. Biotech. 35: 845-851; Jahns et al., 2022, Nucleic Acids Res. 50(3): 1221-1240).

Modification of nucleotides on traditional circRNA is limited because not all of them are compatible with the internal ribosome entry site (IRES). QRNA translation does not require an IRES; thus, is tolerable to more modified nucleotides in a wide range of percentage. These modifications could be spiked into the circular backbone in varying percentages (m6A is typically spiked in at 5%). And the “stem” oligo containing the cap, or the 5′/3′ UTR and tails could likely tolerate a higher percentage of modifications. Alternatively, these modifications can be present in different percentages along different regions of the circular RNA backbone (e.g. in the 5′ UTR, or 3′ UTR, or CDS, or close to the cap structure, or combinations thereof). Furthermore, the “stem” oligo of a Type 1 QRNA (the oligonucleotide containing the cap) is chemically synthesized and could potentially tolerate more complex structures that are difficult to enzymatically incorporate, such as locked nucleic acids (LNAs), 2′ O-methyl nucleotides, peptide nucleic acids (PNAs), morpholinos, and various internal chemical linkers as provided herein.

### Peptides and Polypeptides Encoded by QRNA

Polypeptides encoded by the capped, circularized RNA molecules provided by the invention include any therapeutically useful polypeptide for treatment or intervention of any disease process associated with or dependent on polymorphic or mutant polypeptide species, heritable or acquired as a result of environmental insult or injury. QRNA can encode multiple polypeptides, for example, self-amplifying mRNA cassettes, or multiple therapeutic peptides or polypeptides. In some embodiments, the capped, circular RNA molecule comprises an mRNA region encoding one or a plurality of peptides or polypeptides. A plurality of polypeptides include multiple copies of the same polypeptide or multiple copies of different polypeptides.

An IRES, or self-cleaving peptide such as T2A sequence, can exist between the multiple polypeptide coding sequences on the QRNA. Alternatively, an RNA oligonucleotide containing cap residue site is located before each polypeptide coding sequence, which ultimately will result in a QRNA with multiple cap residue-containing RNA oligonucleotides and ensure that all coding sequences are translated efficiently.

Peptides encoded by capped, circularized RNA molecules of the invention can include but are not limited to therapeutic peptides or antigenic peptides, particularly antigenic peptides suitable for presentation by antigen-presenting cells to humoral (B cells) or cellular (T cells) immune system cells. In certain embodiments these antigenic peptides are adapted to and effective for use as vaccines. In other embodiments the antigenic peptides are adapted to or effective in suppressing immune responses, for example in autoimmune diseases or transplant patients. In additional embodiments the antigenic peptides are adapted to and effective for eliciting specific antitumor immune responses in tumor cells or in attracting cytotoxic native (natural killer cells) or engineered (e.g., CAR-T) cells. Therapeutic peptides encoded by capped, circularized RNA molecules of the invention can include but are not limited to human parathyroid hormone, filgrastim, oxytocin, somatostatin, calcitonin, glucagon, insulin, liraglutide, vasopressin, and the like (see, Fosgerau & Hoffman, 2015, Drug Discovery Today 20:122-128; al Musaimi et al., 2021, Pharmaceuticals (Basil) 14: 145; Wang et al., 2022, Signal Transduct. and Targeted Therap. 7: 1-27).

In some embodiments, peptides encoded by the capped, circular RNA molecules of the invention can include, but are not limited, to Cas9 or derivatives (Rothgangl et al., 2021, Nat. Biotechnol. 39: 949-957) and adenine base editors or other base editors (Gaudelli et al., 2017, Nature 551: 464-471), or RNA base editors for delivery of genome or epigenome editing therapies.

In some embodiments, peptides encoded by the capped, circular RNA molecules of the invention can be selected from any of several target categories including, but not limited to, biologics, antibodies, vaccines, therapeutic proteins or peptides, cell penetrating peptides, secreted proteins, plasma membrane proteins, cytoplasmic or cytoskeletal proteins, intracellular membrane bound proteins, nuclear proteins, proteins associated with human disease, or targeting moieties.

### Synthesis of QRNA

Type 2: The invention also provides methods for producing a type 2 capped, circularized RNA molecules of this aspect of the invention, the methods comprising: synthesizing an RNA oligonucleotide comprising a 5′ end containing a cap structure, an mRNA encoding a peptide or polypeptide, a derivatized nucleotide located between the cap structure and the mRNA region encoding the polypeptide, and a 3′ end containing moiety; and reacting the derivatized nucleotide with the 3′ end moiety to form the covalently linked capped circular RNA molecule.

Type 1: The invention also provides methods for producing a type 1 capped, circularized RNA molecules of this aspect of the invention, the methods comprising the steps of: producing a circularized RNA molecule comprising an mRNA region encoding a peptide or polypeptide, and a derivatized nucleotide outside the mRNA region; synthesizing an RNA oligonucleotide comprising a 5′ end containing cap structure and a 3′ end containing a moiety reactive with the derivatized nucleotide; and reacting the derivatized nucleotide with the 3′ end moiety of the RNA oligonucleotide form a covalently link between the RNA oligonucleotide and the circular RNA. In certain embodiments, the the derivatized nucleotide comprises a moiety that can react with the 3′ end moiety by bioconjugation chemistry, wherein in the bioconjugation chemistry is click chemistry. In addition, the circularized RNA is produced by ribozyme-mediated splicing, enzymatic ligation, or click chemistry-mediated circularization.

Type 3: The invention also provides methods for producing a type 3 capped, circularized RNA molecules of this aspect of the invention, the methods comprising the steps of: producing a circularized RNA molecule comprising an mRNA region encoding a peptide or polypeptide, and a derivatized nucleotide outside the mRNA region; synthesizing an RNA oligonucleotide comprising a 5′ end containing cap structure and a 3′ end containing a moiety reactive with the derivatized nucleotide; and reacting the derivatized nucleotide with the 3′ end moiety of the RNA oligonucleotide form a covalently link between the RNA oligonucleotide and the circular RNA, wherein the synthesis of the circular RNA oligonucleotide, further comprises the steps of: synthesizing an RNA oligonucleotide comprising the mRNA region encoding a peptide or polypeptide, a hairpin structure containing an enzyme-recognition site, and a twister ribozyme sequence on both 5′ and 3′ ends; reacting the RNA oligonucleotide with the enzyme to produce the derivatized nucleotide within the hairpin structure; circularizing the RNA oligonucleotide using the twister ribozyme sequence.

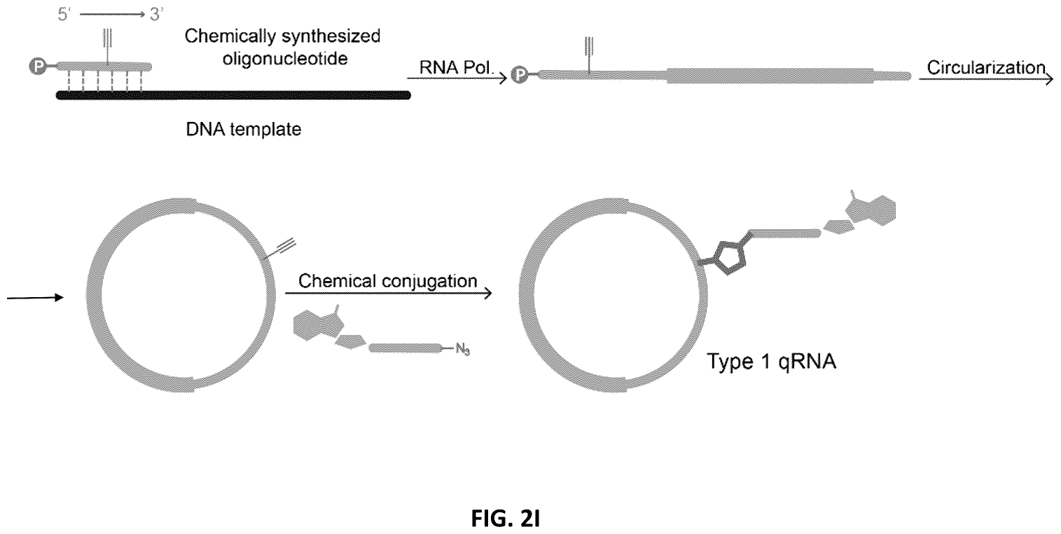

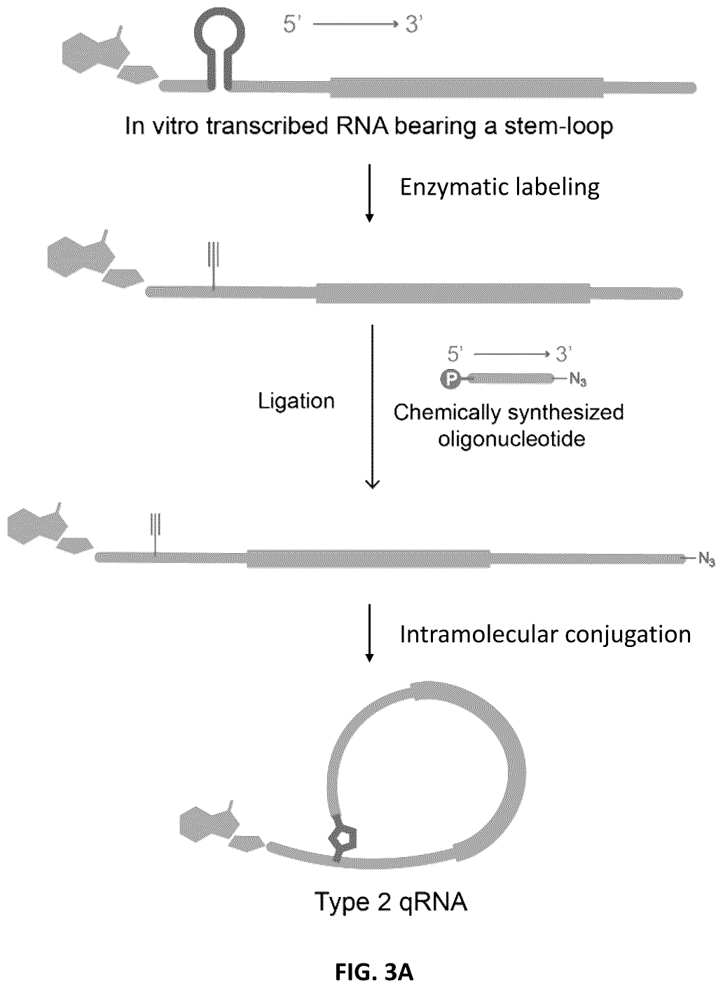

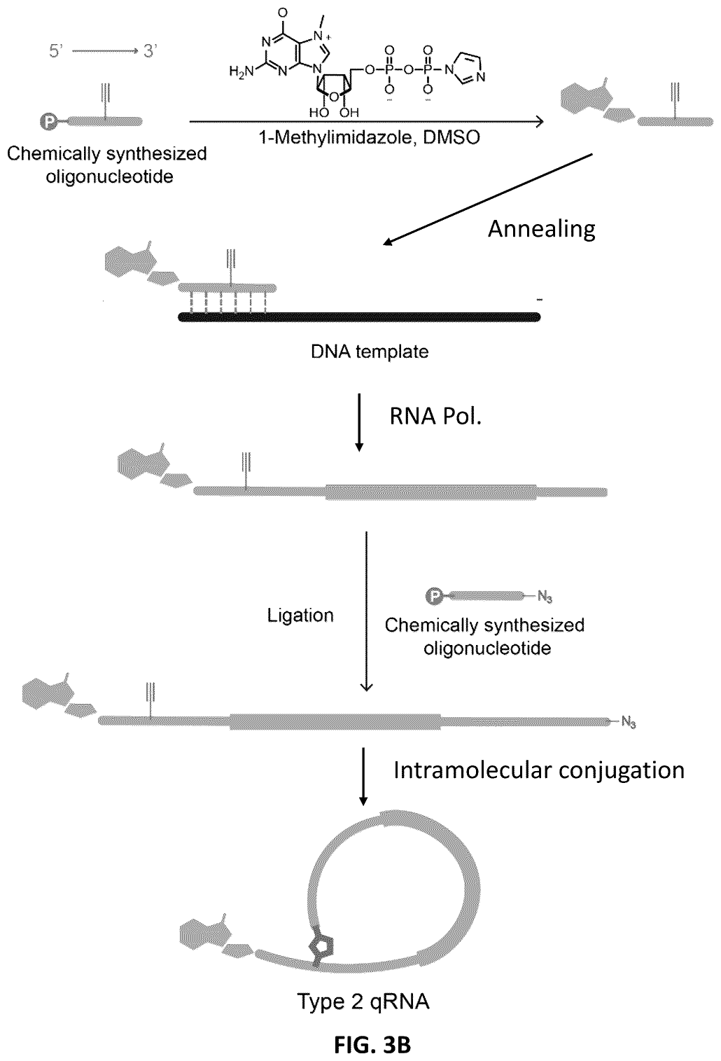

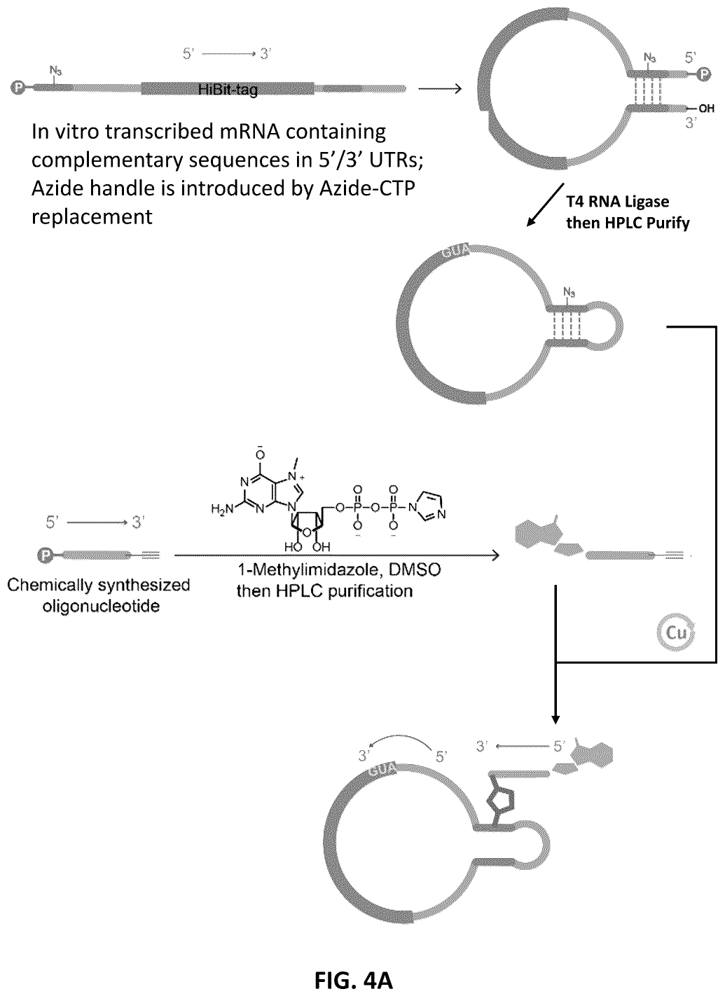

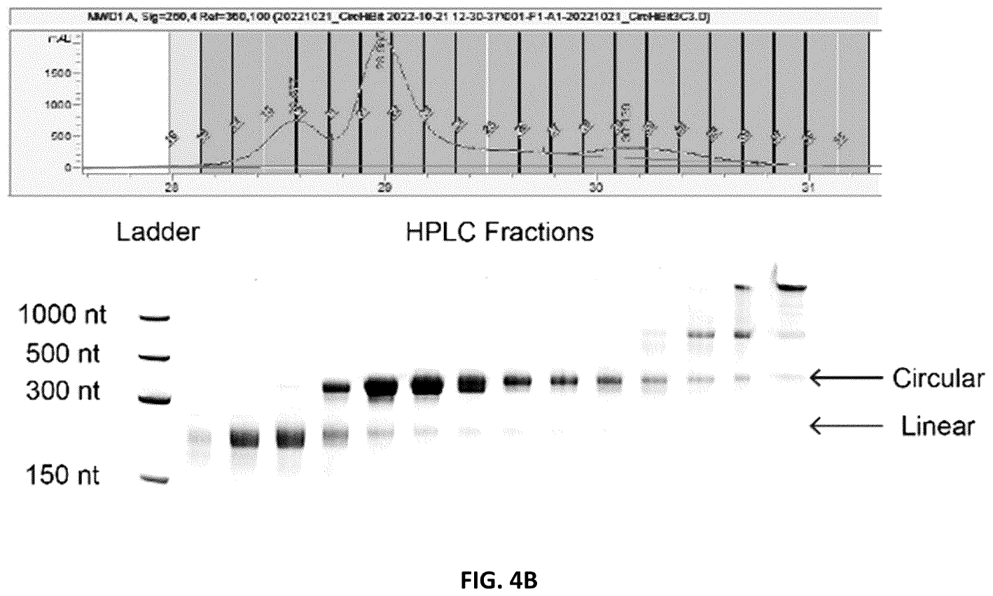

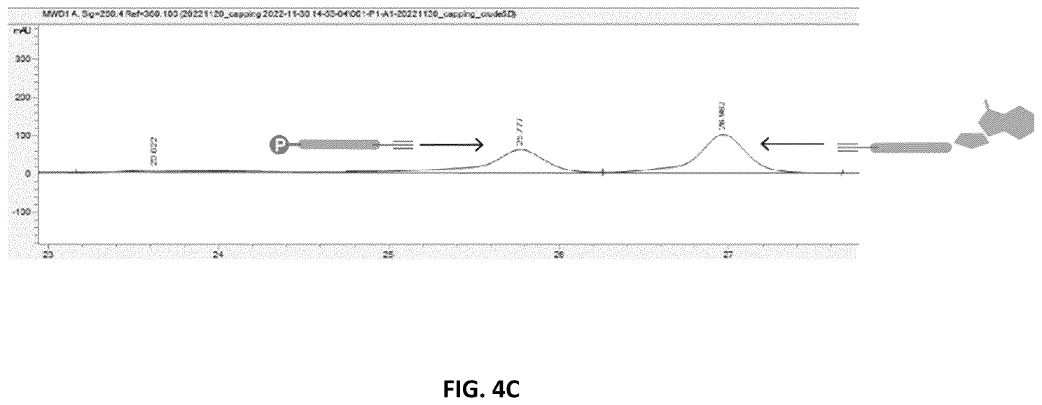

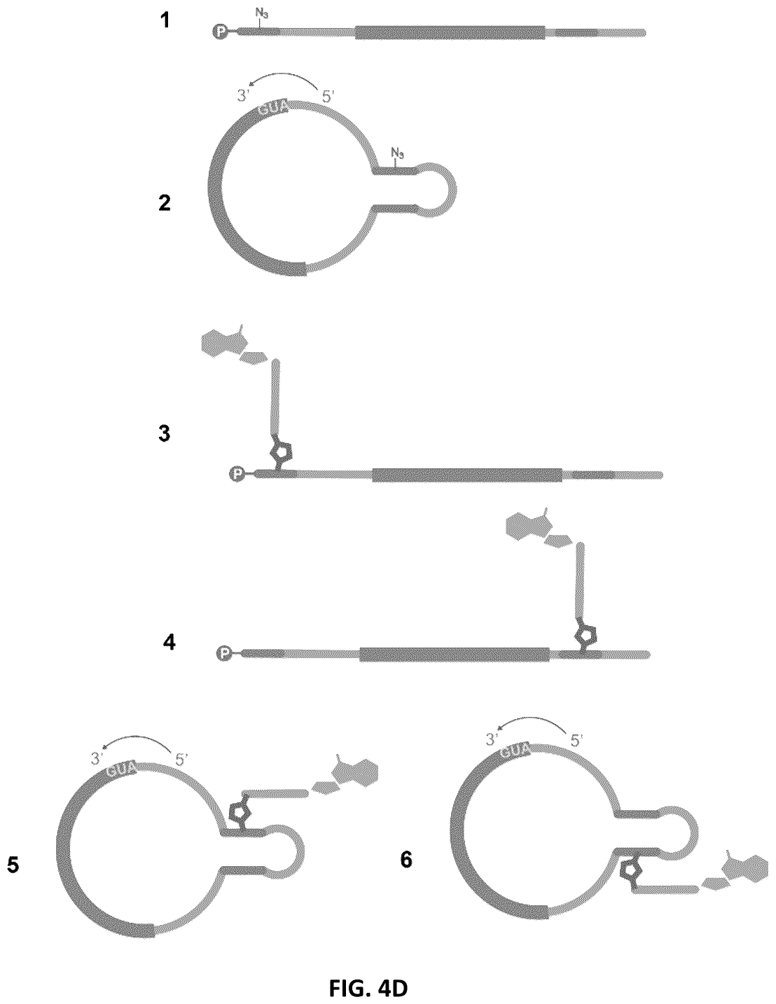

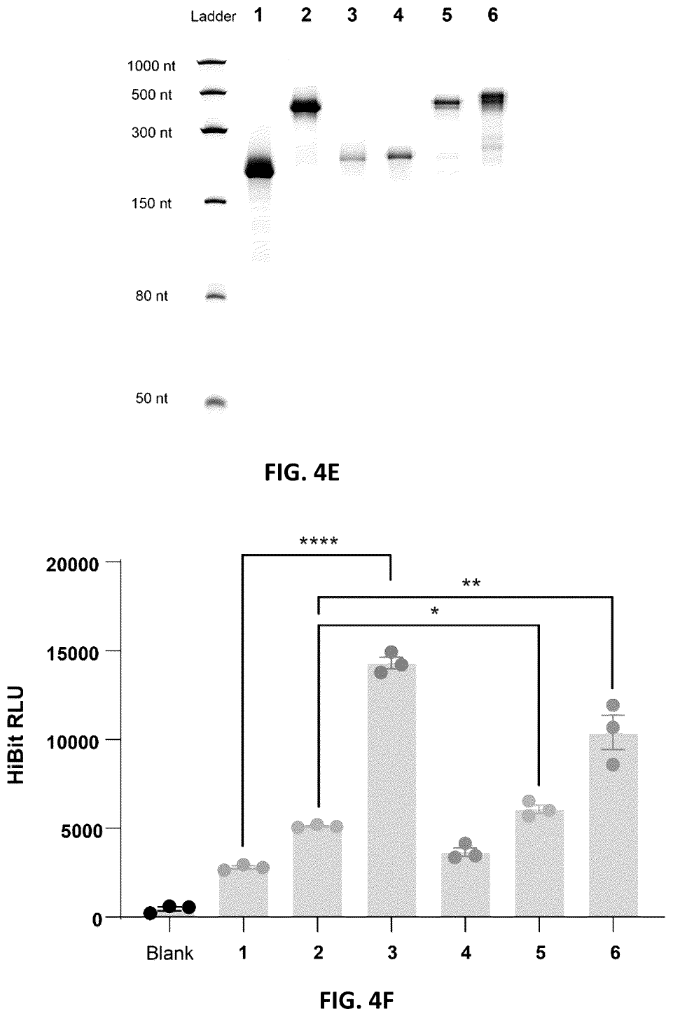

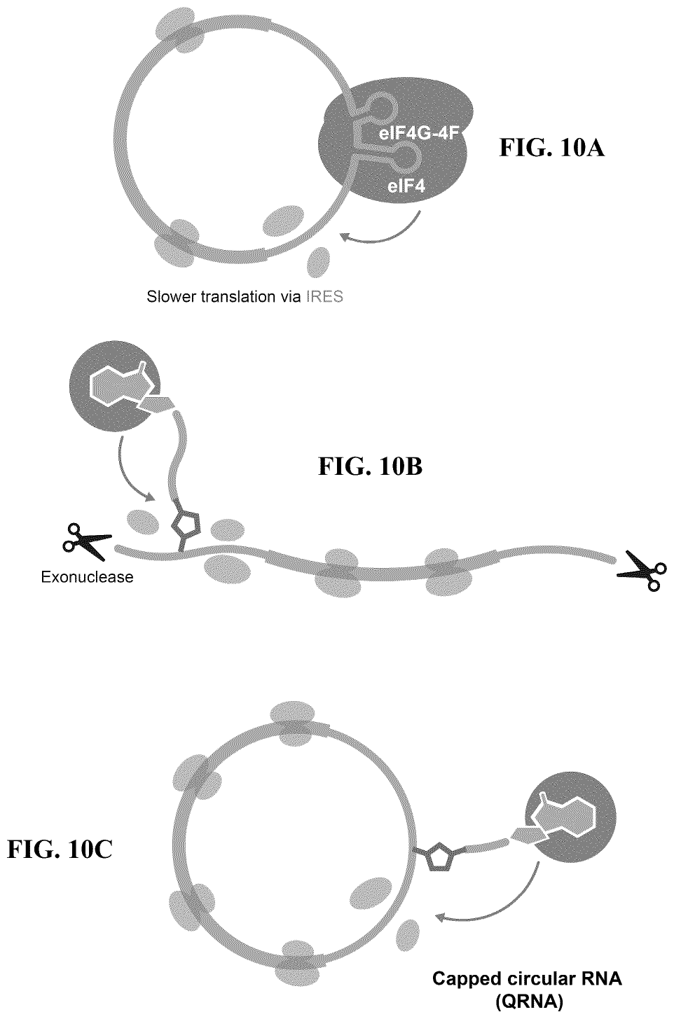

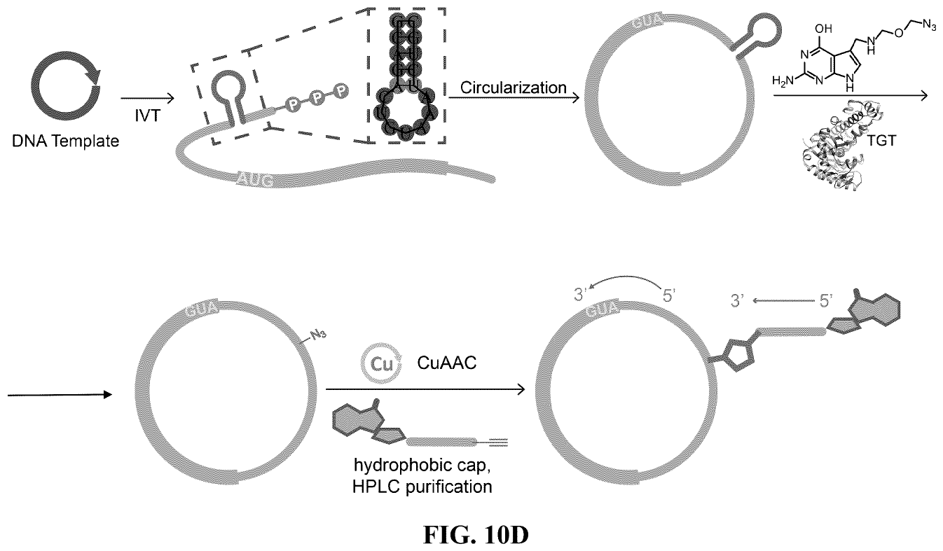

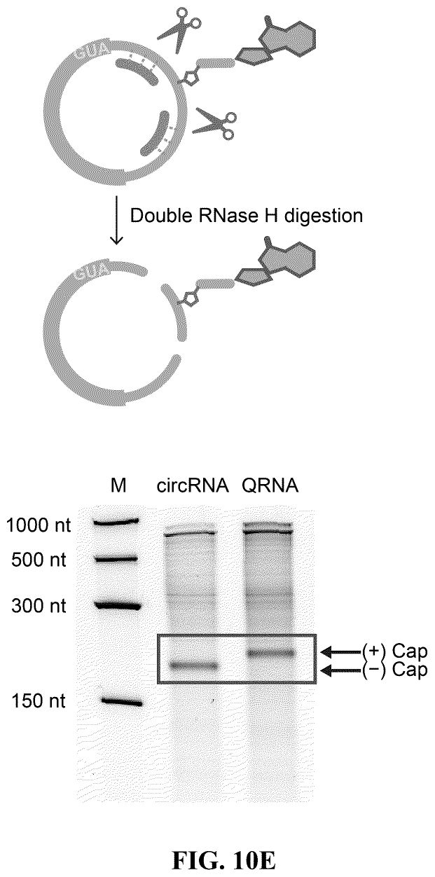

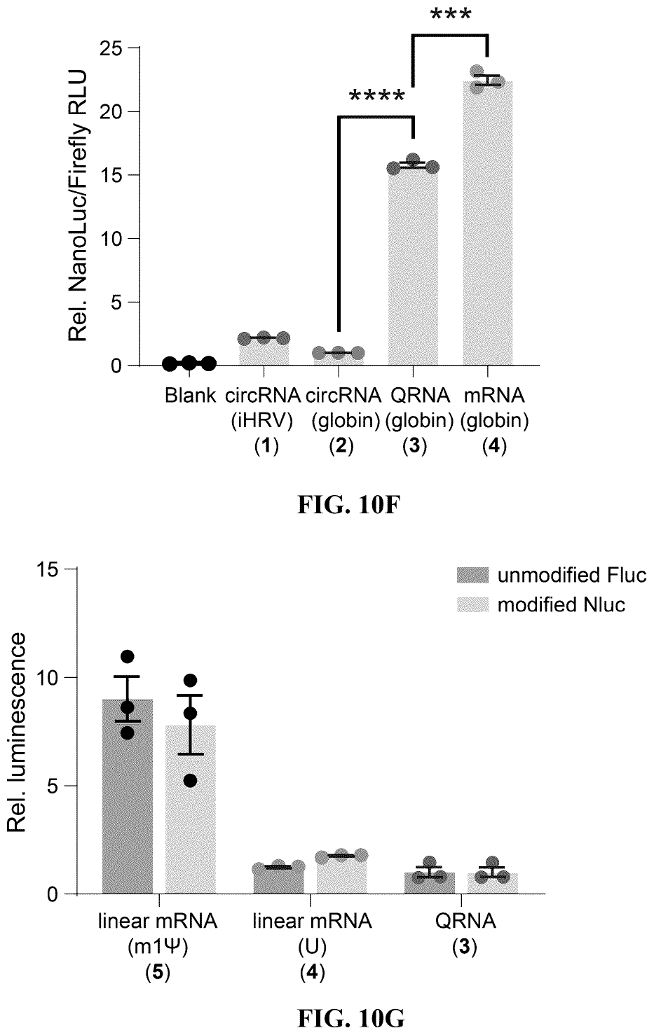

The derivatized nucleotide in these 3 types of QRNA can be generated using different strategies as demonstrated in FIG. 2D-2I, FIG. 4A, and FIG. 10D (type 1), FIG. 3A and FIG. 3B (type 2), and FIG. 1F (type 3). Namely, the derivatized nucleotide can be specifically targeted by having a hairpin structure containing a specific enzyme-recognition site. The enzyme described in some examples in tRNA guanine transferases (TGT). In other examples, the derivatized nucleotide is generated by replacement of a single cytidine with azide-cytidine.

Circularization of RNA molecule in type 1 and type 3 can be achieved by ligation with T4 ligase, RtcB ligase, or ribozyme-mediated splicing. In these embodiments, the 5′ end and 3′ end of the linear oligonucleotide comprise the appropriate moiety to participate in the enzymatic reaction to form the circular RNA. Alternatively, the click chemistry moiety has also been contemplated for the circularization. In some embodiments, additional splint probe containing complementary sequences to the 5′ and 3′ ends of the linear oligonucleotide can be used as demonstrated in the FIG. 2E or FIG. 2G to bring the two ends in proximity and facilitate circularization.

### Pharmaceutical Compositions for Delivery and Methods Therefore

This invention provides pharmaceutical compositions comprising capped, circular RNA molecules of the invention, particularly circularized mRNA molecules. In certain embodiments pharmaceutical compositions of the invention further comprise pharmaceutically acceptable excipients and in certain other embodiments comprise one or more additional therapeutics agents.

In some embodiments, the compositions are suitable to be administered to a human subject in need thereof. In the context of the present disclosure, “active ingredient” refers generally to the capped, circular RNA molecules described herein, particularly circularized mRNA molecules as well as any additional therapeutic agents provided therewith.

It is generally understood by a person of ordinary skill in the art that the compositions described herein are also suitable for administration to any non-human subjects as well. A person of ordinary skill in the veterinary arts will understand that pharmaceutical compositions described herein can be suitable for administration to mammals including but not limited to primates, cattle, pigs, horses, sheep, goats, cats, dogs, mice, rats, whales, and other mammals. A person of ordinary skill in the veterinary arts also will understand that pharmaceutical compositions described herein can be suitable for administration to birds including by not limited to chickens, ducks, geese, turkey, and other domesticated birds, as well as wild birds particularly endangered species of such birds. Additionally, a person of ordinary skill in the veterinary arts will understand that pharmaceutical compositions described herein can be suitable for administration to a wide variety of fish including commercial or wild salmon, tuna, cod, sardine, zebra fish, shark, or the like.

Pharmacological compositions described herein can be prepared by any method known or developed in the art of pharmacology, immunology, virology, or in biotechnology in general.

In some embodiments, the formulations of a pharmacological composition described herein can comprise a unit dose of at least one derivatized RNA, particularly circularized mRNA molecules, in addition to at least one other pharmaceutically acceptable excipient. Such excipients can include but are not limited to, solvents, dispersions, buffers, diluents, surfactants, emulsifiers, isotonic agents, preservatives, thickeners, lubricating agents, oils, or the like.

In some embodiments, the pharmacological composition can comprise a delivery mechanism further comprising a lipid nanoparticle. The size of the lipid nanoparticle can be altered to counteract immunogenic response from the subject, or to allow for increased potency and pharmacological activity.

In other embodiments, the pharmacological composition can comprise a delivery mechanism further comprising a lipidoid as previously described in the art. See Akinc et al., 2008, Nat Biotechnol. 26:561-596; Frank-Kamenetsky et al., Proc Natl Acad Sci USA. 2008 105:11915-11920; Akinc et al., 2009, Mol Ther. 17:872-879; Love et al., 2010. Proc Natl Acad Sci USA 107:1864-1869; Leuschner et al., 2011, Nat Biotechnol. 29:1005-1010, all of which is incorporated herein in their entirety. Lipidoids refers broadly to lipid nanoparticles, liposomes, lipid emulsions, lipid micelles and the like. Lipidoids containing the pharmacological composition comprising the derivatized RNA can be administered parenterally by means including but not limited to, intravenous injection, intramuscular injection, subcutaneous injection, via dialysate, intrathecal injection, or intracranial injection.

A person of ordinary skill in the art would also recognize that other nucleotide delivery mechanisms exist such as the use of viral like, or viral derived particles. See Rohovie et al., 2016, Bioengineering & Translational Med. 2(1): 43-57. Virus like particles can include coat proteins or viral capsids of a virus. Such particles can be PEGylated or further annealed to compounds that avoid phagocytotic clearance. Additionally, the surface of the virus like particle can be further functionalized to provide cellular specific targeting, facilitate extravasation, facilitate radio labeling, improve permeability across cellular boundaries, or to transcytose the blood-brain barrier. The virus like particles can be derived for animal viruses, bacteriophages, or plant viruses. Examples of suitable virus for derivation of a virus like particle delivery mechanism include but are not limited to cowpea chlorotic mottle virus, cowpea mosaic virus, hepatitis B virus (core), enterobacteria phage MS2, Salmonella typhimurium P22, enterobacteria phage Qβ amongst other suitable viruses. Derivatized RNA payloads can be loaded into the virus like particles by electrostatic adsorption or any other suitable method known to a person of ordinary skill in the art.

Various exemplary embodiments of compositions and methods according to this invention are now described in the following non-limiting Examples. The Examples are offered for illustrative purposes only and are not intended to limit the scope of the invention in any way. Indeed, various modifications of the invention in addition to those shown and described herein will become apparent to those skilled in the art from the foregoing description and the following examples and fall within the scope of the appended claims.

### EXAMPLES

### Example 1. Synthesis of Type 1 Capped Circular RNA

Synthesis of type 1 QRNAs can be divided into two principal steps: synthesis of 5′-capped oligonucleotides bearing a 3′-click chemistry handle; and synthesis of circular RNA containing corresponding click chemistry handles in its untranslated regions (“UTRs”, i.e. outside the portions of the RNA encoding inter alia a protein or peptide). The 5′-capped oligo can be synthesized from an oligonucleotide generated by solid phase synthesis (with click handles and 5′-phosphate incorporated therein), followed by chemically capping the RNA using N7-methylated GDP imidazolide (FIG. 2B) (Abe et al., 2022, ACS Chem. Biol. 17: 1308-1314) or enzymatically capped using capping enzymes such as the Vaccina capping enzyme with guanine methyl transferases (FIG. 2C) (Shuman in Progress in Nucleic Acid Research and Molecular Biology, 50: 101-129 (Cohn & Moldave, Eds. Academic Press, 1995; Kyrieleis et al., 2014, Structure 22: 452-465). CircRNAs containing click chemistry handles were synthesized in multiple ways. In non-limiting examples, a linear mRNA with click handles in its 5′/3′-UTRs was generated through chemical or enzymatic synthesis, and this transcript was designed in a way that it contained complementary sequences in 5′/3′-UTRs to allow circularization using single-stranded RNA ligases (T4 RNA ligase) (FIG. 2D) (Wang and Ruffner, 2014, Nucleic Acids Res. 26: 2502-2504). An example of QRNA constructed according to the strategy depicted in FIG. 2D was carried out as shown in FIG. 4A and Example 4 below. Alternatively, a click handles-containing transcript can be circularized using a splint probe complementary to sequences on the 5′/3′-ends, allowing circularization using double-stranded RNA ligases such as T4 RNA ligase II (FIG. 2E) (Chen et al., 2020, Nucleic Acids Res. 48: e54). Circular RNA with click handles can also be generated through tandem ligation process: an RNA transcript containing 5′/3′-hydroxyl groups can be synthesized through in vitro transcription (IVT), and ligate to a chemically synthesized oligonucleotide that has click chemistry handles and phosphate groups on both 5′- and 3′-ends. The 5′-phosphate/3′-hydroxyl ends can be ligated by T4 RNA ligase and the 5′-hydroxyl/3′-phosphate ends can be ligated using RNA ligase RctB to yield the desired circRNA. The ligation reaction can also be splint-guided to improve yields (FIG. 2G). Alternatively, the RNA transcript can be made with in vitro transcription and bears 5′-triphosphate and 3′-hydroxyl. A chemically synthesized oligonucleotide having 5′-phosphate, 3′-phosphate, and the derivatized nucleotide is ligated to the mRNA first using T4 RNA ligase (linking mRNA 3′-hydroxyl to the oligonucleotide 5′-phosphate). Then the mRNA 5′-triphosphate is hydrolyzed using calf intestinal alkaline phosphatase (CIAP) to a 5′-hydroxyl. Then the mRNA is circularized by ligating the 5′-hydroxyl and 3′-phosphate using RtcB ligase (FIG. 211). Rather than incorporating the click handle before/during RNA circularization, an RNA bearing a stem-loop motif can be synthesized through IVT and circularized through ribozyme-mediated backsplicing (Wesselhoeft et al., 2018, Nat Commun. 9: 2629), and the click handle can be introduced with hairpin modifying enzymes such as the tRNA guanine transferases (TGT) (Alexander et al., 2015, J. Am. Chem. Soc. 137: 12756-12759) (FIG. 2F).

Another way to combine cotranscriptional circularization and click handle incorporation is to use a synthetic oligonucleotide containing the click handle as a primer to anneal onto the DNA template, and in vitro transcribed using an engineered DNA polymerase that synthesize RNA from primers (Cozens et al., 2012, Proc. Natl. Acad. Sci. U.S.A 109, 8067-8072), followed by ligation/backsplicing-based circularization (FIG. 2I). Upon successful synthesis, the circRNA and capped oligo are crosslinked using click chemistry to yield the type 1 circRNA. As an alternative to the aforementioned strategies, a circular mRNA scaffold can be constructed by chemical circularization (e.g. an RNA circularized via a nonnative/non-phosphodiester chemical linkage), such as by incorporation of click handles on the 5′ and 3′ ends of a linear RNA. After successful chemical circularization, this circular scaffold is then further modified by the covalent addition of a 7-methylguanosine cap or a cap-containing oligonucleotide.

### Example 2. Synthesis of Type 2 Capped Circular RNA

For type 2 QRNA, a 5′-capped mRNA transcript containing an RNA hairpin in 5′-UTR can be synthesized by IVT and co-transcriptional capping, and a first click chemistry handle (e.g., an alkyne) can be introduced using hairpin labeling enzymes such as TGT. Subsequently, the second click chemistry is introduced by ligating a chemically synthesized oligo having 3′-terminal azide handle to the 3′-end of the transcript, and the mRNA can be intramolecularly circularized using click chemistry to yield type 2 QRNA (FIG. 3A). Alternatively, a synthetic oligonucleotide containing the click handle can be used as a primer to anneal onto the DNA template, and transcribed in vitro using an engineered DNA polymerase that synthesizes RNA from primers (Cozens et al., 2012, Proc. Natl. Acad. Sci. U.S.A 109: 8067-8272; Freund et al., 2023, Nat. Chem. 15:91-100). The IVT product is subsequently ligated to the 3′-azide/5′-phosphate synthetic oligo, allowing intramolecular chemical circularization (FIG. 3B)

### Example 3. Synthesis of Type 3 Capped Circular RNA

Description of synthesis strategy: The synthetic scheme for Type 3 QRNA is set forth in FIG. 1F to FIG. 1H. Short QRNAs encoding a FLAG peptide (DYKDDDDK—SEQ ID NO: 1, where D=aspartic acid, Y=tyrosine, and K=lysine) are prepared and tested. The 3×FLAG peptide (DYKDDDDKDYKDDDDKDYKDDDDK—SEQ ID NO: 2) having the sequence set forth herein can be replaced or re-coded into other reporter peptides (e.g. NanoBiT 11-amino acid peptide, as shown at www.promega.com/products/protein-interactions/live-cell-protein-interactions/nanobit-ppi-starter-systems/?catNum=N2014) or a therapeutic peptide (e.g. for peptide-based vaccines).

Oligo 3.1 is generated with in vitro transcription and circularized co-transcriptionally (see, Litke et al., 2019, Nature Biotechnol. 37: 667-675) by the twister ribozyme and RNA ligase RtcB to produce circular RNA 3.2. Circular RNA 3.2 is then labeled post-transcriptionally and site-specifically using tRNA guanine transglycosylase (TGT) and a synthetic preQ1 cofactor analogue (FIG. 1G) to incorporate a 5-methyl-tetrazine handle at a tRNA-like hairpin structure (see, Alexander et al., 2015, J. Amer. Chem. Soc. 137: 12756-127592015)) to produce circular RNA 3.3. Oligonucleotide 3.4 is prepared similarly as described in Example 1 by replacing 3-Azide-2,3-ddUTP with 5-TCO-PEG4-dUTP to produce a capped oligo with 3′-TCO handle (FIG. 111). Oligo 3.4 is then ligated onto the circular RNA 3.3 via tetrazine-TCO cyclization to yield the desired 3×Flag-encoding Type 1 QRNA 3.5. Sequences of oligonucleotides 3.1 and 3.4 are presented in Table 1.

TABLE 1
Sequences of Synthetic Oligonucleotides
used in design of QRNA Type 3

| Oligo | Sequence |
|---|---|
| 3.1 | AAGAGCGAGCTCTAATACGACTCACTATAGGGGCCGCACTCGCC |
| | GGTCCCAAGCCCGGATAAAATGGGAGGGGGCGGGAAACCGCCTA |
| | ACCATGCCGAGTGCGGCCGCAAAAAAGCAGACTGTAAATCTGAA |
| | AAAAGAGAAUAAACUAGUAUUCUUCUGGUCCCCACAGACUCAGA |
| | GAGAACCCGCCACCAUGGACUACAAAGACGAUGACGACAAGAUG |
| | GACUACAAAGACGAUGACGACAAGAUGGACUACAAAGACGAUGA |
| | CGACAAGUAGAAAAAAAAAAAAAAAAAAAAAAAAAAAAAAGTGG |
| | CCGCGGTCGGCGTGGACTGTAGAACACTGCCAATGCCGGTCCCA |
| | AGCCCGGATAAAAGTGGAGGGTACAGTCCACGCTCTAGATATAG |
| | CCTTATAGCCT |
| | (SEQ ID NO: 3) |
| 3.4 | [symbol] AAACAAAACAAAACAACCAACAACAACACAACAAU |
| | (SEQ ID NO: 4) |

Note:
bases listed are all ribonucleotides unless specified
otherwise. [symbol]
= m7G(5′)ppp(5′)(2′OMeA)pG (e.g. ″CleanCap AG″ 5′ cap
analogue from TriLink Biotechnologies); U =
5-TCO-PEG4-2-deoxyuridine; U = N1-methylpseudouridine or
uridine

### Example 4. Type 1 Capped-Circular mRNA has Higher Translation Efficiency than Uncapped-Circular mRNA

QRNA construct using a small RNA was used, for ease of synthesis, purification, and characterization. The RNA template (FIG. 4A) contained 5′ and 3′ complementary regions (shown in dashed line) to facilitate annealing and subsequent circularization (e.g. enzymatic ligation of the 5′ and 3′ ends). This RNA scaffold contained a short coding sequence encoding a 12 amino acid HiBit tag (shown in blue; MVSGWRLFKKIS—SEQ ID NO: 5), as well as a short poly(A) region following the coding sequence (CDS) to mimic the architecture of linear mRNA. Either the 5′ UTR or 3′ UTR contained a single C site for the installation of cytidine triphosphate (CTP)-azide during transcription. This single CTP-azide incorporation allowed us to click a 5′ 7-methylguanosine cap oligo onto the circularized RNA scaffold.

The RNA scaffold containing a 5′ C-azide site had the following sequence (5′ to 3′):

(SEQ ID NO: 6)
ggaaaaaaaaaagaaaaaaaagaauaaauuuCuauuauuaauauaauuaa
auuaaaauuaagagaagggaagauggugaguggauggagauuauuuaaga
agauuaguuaguaauuuuaauuuaauuauauuaauaauagaaauuuaaaa
aaaaaaaagaaaaaaaaaaaaaaaaaaaaaaaa.

The RNA scaffold containing a 3′ C-azide site had the following sequence (5′ to 3′):

(SEQ ID NO: 7)
ggaaaaaaaaaagaaaaaaaagaauaaauuuguauuauuaauauaauuaa
auuaaaauuaagagaagggaagauggugaguggauggagauuauuuaaga
agauuaguuaguaauuuuaauuuaauuauauuaauaauaCaaauuuaaaa
aaaaaaaagaaaaaaaaaaaaaaaaaaaaaaaaa.

The above sequences served as scaffolds for “circular” coding portion of the tested mRNA. The underlined 5′ and 3′ regions were complementary and facilitated enzymatic circularization of the RNA by enzymatic ligation. The bold text shows the protein-coding sequence encoding a HiBit tag (MVSGWRLFKKIS*—SEQ ID NO: 5), where * is the stop codon. Additionally, the single C site (shown in bold capital text) was encoded by this template and was located in either the 5′ or 3′ complementary region. A site-specific azide functionalization in the circular RNA scaffold was achieved by only encoding a single cytidine in these templates (FIG. 4A).

The above RNAs were synthesized by in vitro transcription (IVT) from a corresponding DNA template, using the following nucleotide triphosphates as precursors: GTP, ATP, 5-azido-PEG4-CTP (Jena Bioscience, Catalogue Number: CLK-0523) instead of normal CTP, and N1-methylpseudo-UTP (Jena Bioscience, Catalogue Number: NU-890) instead of UTP. All internal instances of cytidine were removed except for the single sites (shown in capital bold text), to prevent nonspecific azide incorporation within the coding region or at other sites within these templates.

RNAs produced by IVT contained a 5′ triphosphate and 3′ hydroxyl group. Following IVT synthesis, RNA was treated with RNA 5′ Pyrophosphohydrolase (RppH) (New England BioLabs, Catalogue Number: M0356S) to produce a 5′ phosphate. Subsequently, these RNAs were annealed and T4 RNA ligase 1 was used (New England BioLabs, Catalogue Number: M0437M) to ligate the 5′ and 3′ ends together to produce a circular “scaffold” that would serve as the base of the QRNA structure (FIG. 4A). Circularized RNA was separated from contaminants using high-performance liquid chromatography (HPLC) purification (FIG. 4B), and used as a subsequent scaffold for click functionalization.

An oligo containing a 5′ 7-methylguanosine cap and a 3′ alkyne group was synthesized (FIG. 4A and FIG. 4C). Synthetic RNA oligonucleotide bearing 5′-phosphate and 3′-alkyne was generated through solid phase synthesis (ordered with IDT, sequence order code: /5Phos/rArGrArArUrArA/35OCTdU/). Synthetic oligos having ammonia as counterions were dissolved in DMSO, treated with 100 equivalents of m7GDP-imidazolide in 4% (v/v) 1-methyl-imidazole in DMSO at 55° C. for 3 hours, and HPLC purified. This 5′ cap oligo was covalently linked to the circular RNA scaffold using copper-catalyzed azide-alkyne click chemistry (FIG. 4D).

For expression tests, QRNA expression was compared to various precursors, including a cap-oligo covalently linked to a linear template, or a circular template not containing a cap (FIG. 4D). QRNA and relevant synthetic precursors (shown as 1-6 in FIG. 4D) were transfected to HeLa cells using lipofectamine (ThermoFisher Scientific, Catalogue Number: LMRNA001) and bioluminescence was measured 8 hrs post transfection using Hibit lytic assay (Promega, Catalogue Number: N3030). A significant enhancement of translation was observed when the linear precursor was conjugated to the capped oligo on the 5′-end but not on the 3′-end, indicating that the triazole-linked cap induced translation efficiency (TE) enhancement (1, 3, 4 shown in FIG. 4F). Similarly, increased luminescence was achieved when the circular RNA was conjugated to the capped oligo, in which case the more upstream click handle in the original 3′-UTR outperformed the one in the more proximal 5′-UTR (2, 5, 6 as shown in FIG. 4E), confirming QRNA as a feasible strategy for improving circRNA translation.

### Example 5. Crosslinking Reactions Contemplated for Synthesis of Capped-Circular RNA

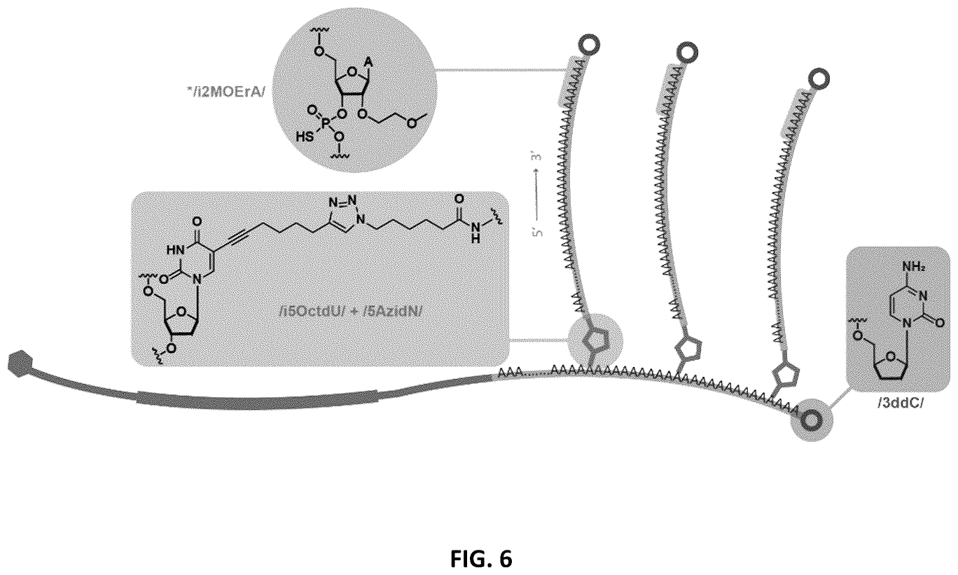

Experiments described in this disclosure demonstrate synthesis of branched poly(A) oligonucleotides mRNA as shown in FIG. 6. Similar conditions described herein can be applied to chemical conjugation and intramolecular conjugation in synthesis of type 1 and type 2 QRNA respectively. Chemically modified poly(A) oligonucleotides were obtained from Integrated DNA Technologies and suspended in RNase-free water to 100 uM final concentration.

For thiol-ene/yne conjugation (an organic reaction between a thiol (R—SH) and an alkene (R2C═CR2) or alkyne to form a thioether (R—S—R′) with chemical structures demonstrate in FIG. 5A), disulfide-protected thiol-oligos were deprotected with 100 molar excess of TCEP (tris(2-carboxyethyl)phosphine) and immediate mixed with equal molar of alkene/alkyne modified oligos and incubated at 37° C. for 30 mins to 1 hr. In cases where radical conditions were used, substoichiometric amounts of 2,2-dimethoxy-2-phenylacetophenone were added and incubated at room temperature under 370 nm radiation (Kessil, Catalogue Number: KSPR160L370). The size of the products and precursors of crude thiol-ene/yne oligonucleotide conjugation of 15-nt model substrates containing only one conjugation handle are shown in FIG. 5C.

For amine-phosphate conjugation (top panel, FIG. 5B), oligos were mixed in excess amounts of imidazole with 1-2 equivalents of EDC as an additive. The reaction mixture was incubated at 37° C. for 30 mins to 1 hr.

For IEDDA (inverse demand Diels-Alder reaction) conjugation, methyl tetrazine (Me-Tz) and transcycle-octene (TCO) labeled oligos were obtained from corresponding amine-modified oligos upon labeling with Tetrazine-PEG5-NHS Ester (Click Chemistry Tools, Catalogue Number: 1143) or TCO-PEG4-TFP Ester (Click Chemistry Tools, Catalogue Number: 1198) overnight at 4° C. in 100 mM NaHCO₃ with a molar ratio of 500:1 (small molecule: oligonucleotides). NHS-labeling products were purified using ethanol precipitation. Me-Tz/TCO-labeled oligos were suspended in RNase-free water and incubated at 55° C. for 30 mins.

For CuAAC (copper-catalyzed azide-alkyne conjugation) conjugation, azide/alkyne-containing oligonucleotides were mixed with indicated molar ratio (1:1 for one-branched oligo, 2:1 for di-branched oligo, and 3:1 for tri-branched oligo). The size of the products and precursors of CuAAC and IEDDA 30-nt oligonucleotides bearing three EU/TCO handles reacting with 30-nt N3/Tz modified oligo are shown in FIG. 5D. Branched oligo structures are shown in FIG. 6. The oligonucleotide mix was diluted in a modified 1.5× click chemistry buffer (Lumiprobe, Catalogue Number: 61150, with 5% SUPERase Inhibitor, 5% DMSO, and 5% 10 mM dNTP mix [ThermoFisher Scientific, Catalogue Number: 18427089]) that was briefly de-gassed by argon purging for 20 mins prior to the reaction. For a typical 100 μL reaction, 33 μL of oligonucleotide solution was mixed with 66 μL of click chemistry buffer and 2 μL of 100 mM L-ascorbic acid solution (Sigma Aldrich, Catalogue Number: A5960) was added immediately prior to the reaction. The mixture was incubated at 37° C. for 1 hr and the reaction was stopped by addition of 1 μL of 500 mM EDTA (pH 8.0). The reaction was first purified using Monarch RNA Cleanup Kit (NEB, Catalogue Number: T2040) and the crude products were repurified using RNase-free HPLC on an Agilent 1260 Infinity II HPLC with acetonitrile [Sigma Aldrich, 34851] and 100 mM hexylamine/acetic acid (pH 7.0, with 10% urea w/v) as mobile phase. HPLC fractions were analyzed by Novex TBE Urea gels, stained by 1×SYBR Gold (ThermoFisher Scientific, Catalogue Number: S11494), and visualized using BioRad ChemiDoc MP Imaging System (Catalogue Number: 12003154). Desired fractions were then pooled, desalted, and concentrated using Monarch RNA Cleanup Kit for small-scale preparations or ethanol precipitation for large-scale preparations.

### Example 6. Covalent Internal Cap Drives Robust Translation in Linear and Circular RNAs

Systematic Interrogation of 5′-Modifications on mRNA Translation

Traditional approaches of preparing capped therapeutic mRNA include enzymatic or co-transcriptional capping. For enzymatic capping, mRNA transcript is treated with capping enzymes and methyltransferases post in vitro transcription (IVT) (Ramanthan et al., 2016, Nucleic Acids Res. 44: 7511-7526). Co-transcriptional capping is achieved by spiking synthetic cap analogues into the IVT reaction. The first generation of dinucleotide cap analogue m⁷G(5′)ppp(5′)G would result in unintended “reversed” orientation of the m⁷G cap, and this issue was solved by adoption of the anti-reverse cap analogue (ARCA) (Grudzien-Nogalska et al., in Methods in Enzymology (Academic Press, 2007), 431: 203-227; Stepinski et al., 2001, RNA 7: 1486-1495). Recent developments of trinucleotide/tetranucleotide cap analogues for direct incorporation of Cap-1/Cap-2 structures and most cap modifications were screened using tri/tetranucleotides (Ishikawa et al., 2009, Nucleic Acids Symp Ser. 53: 129-130; Sikorski et al., 2020, Nucleic Acids Res. 48: 1607-1626; Jurga et al., Messenger RNA Therapeutics (Springer Nature, 2022)). However, neither method accesses modifications beyond the first two bases and leads to potential bias in screening, due to different incorporation efficiencies of various cap structures. Additionally, purification of capped mRNAs from uncapped ones could not be easily achieved given the similar physicochemical properties of both species.

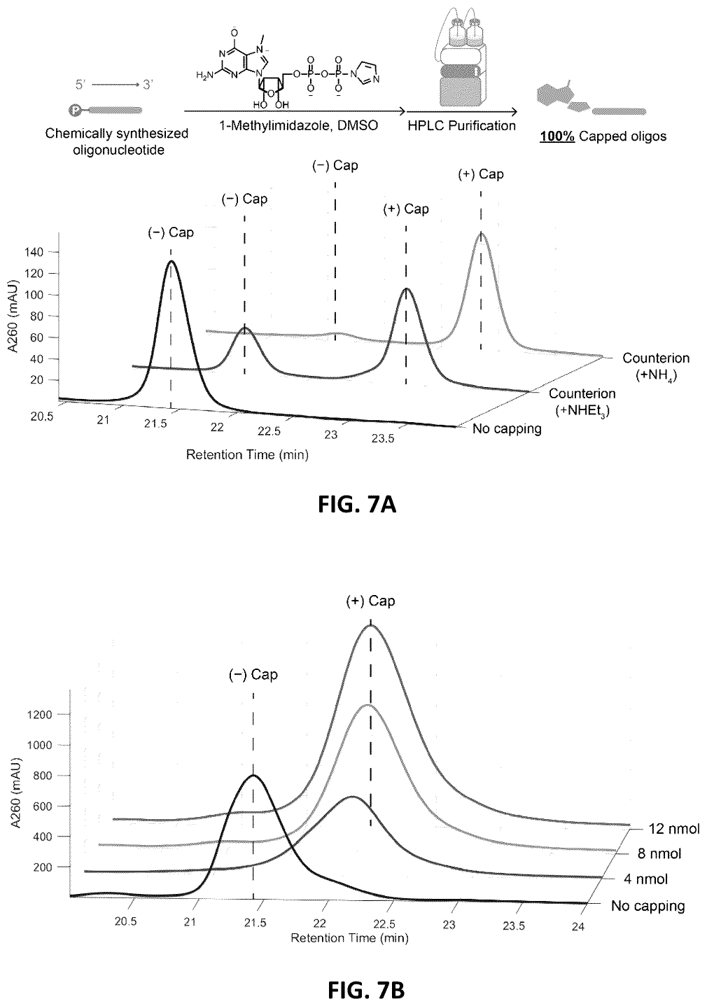

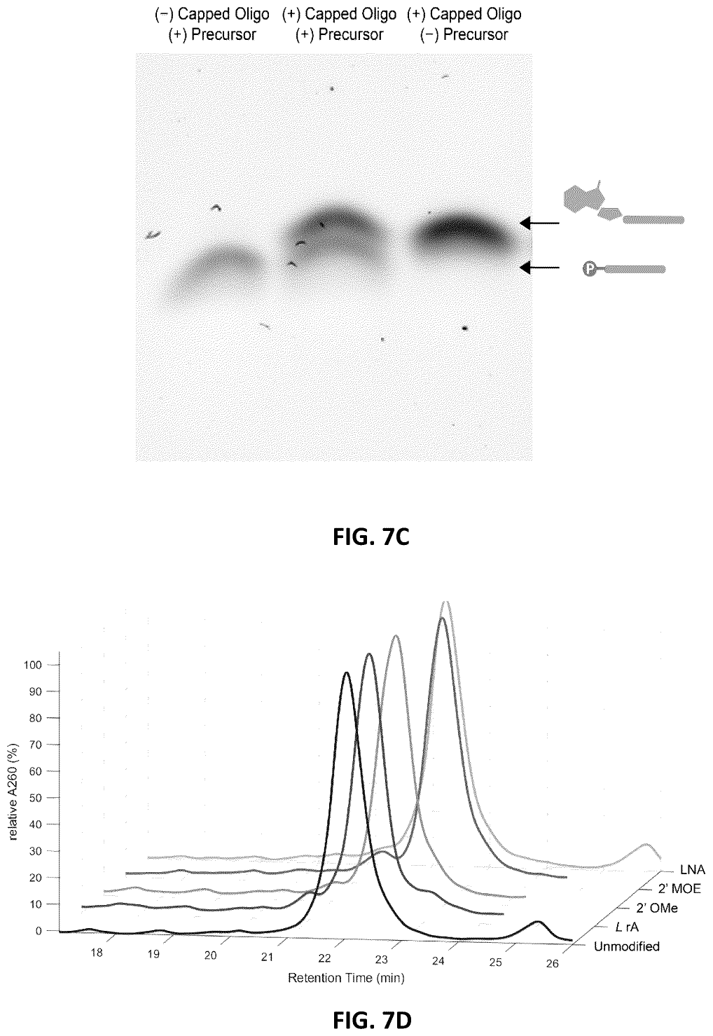

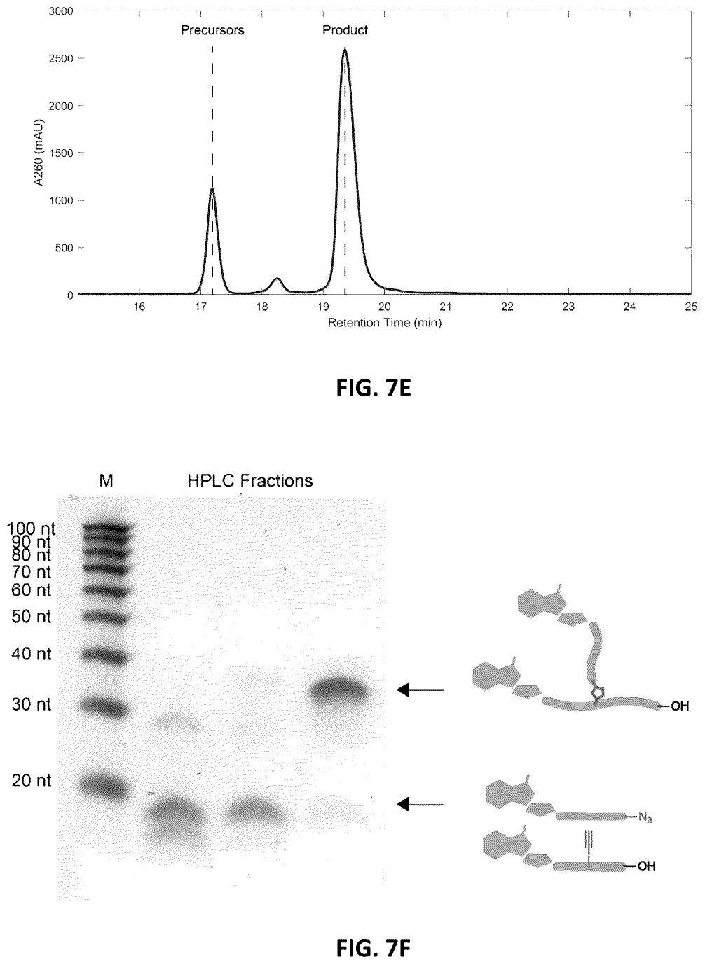

To solve these challenges, the capping step was separated from mRNA synthesis. Oligonucleotides with defined chemical modifications were facilely synthesized on solid phase, which was subsequently capped chemically using m⁷G diphosphate imidazolide (m⁷GDP-Im) derivatives (Abe et al., 2022, ACS Chem. Biol. 17: 1308-1314). Altering the oligonucleotide counterion to ammonium allowed robust capping without any divalent ion additives, and fine-tuning of reversed-phase high-performance liquid chromatography (RP-HPLC) gradient with more hydrophobic hexyl ammonium ions enabled isolation of 100% capped product at scale (FIG. 7A to FIG. 7D). These capped oligos were subsequently ligated to 5′-monophosphorylated mRNAs containing N¹-methylpseudouridine (m¹Ψ) generated from IVT followed by bacterial RNA 5′ pyrophosphohydrolase (RppH) treatment.

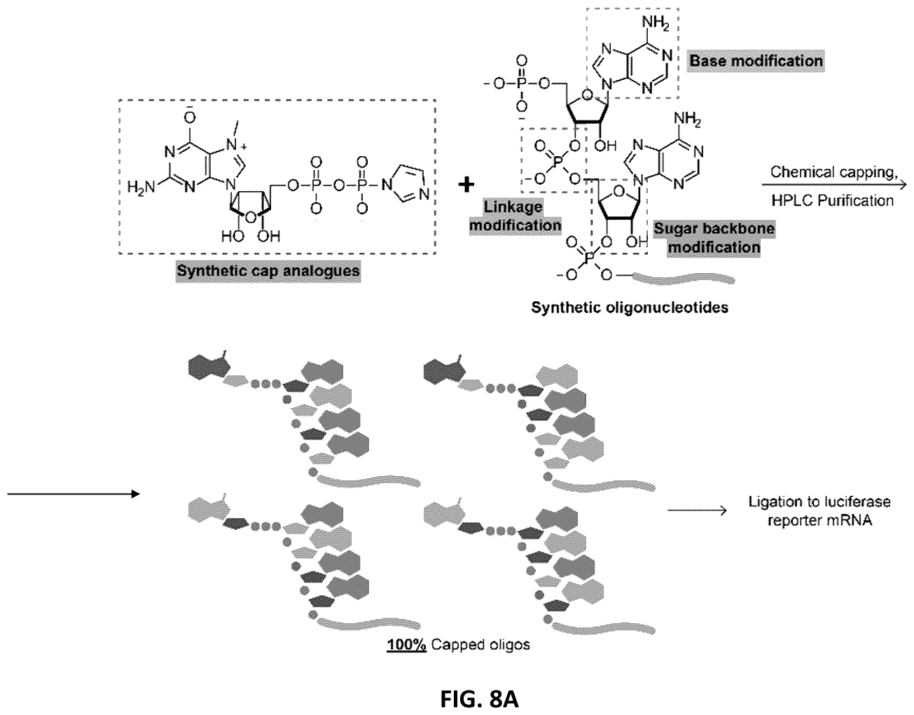

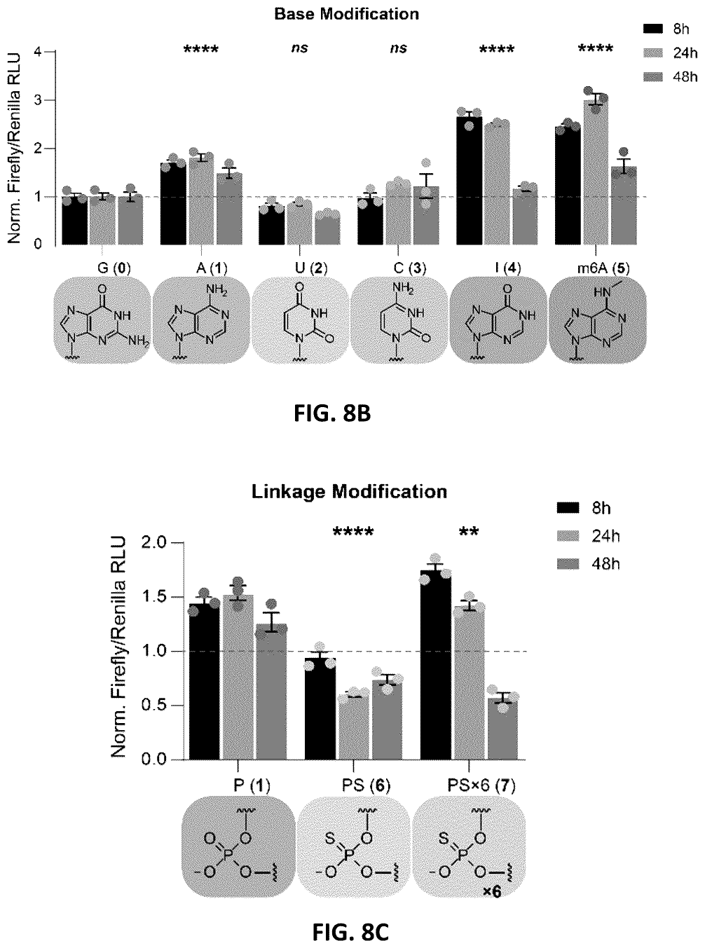

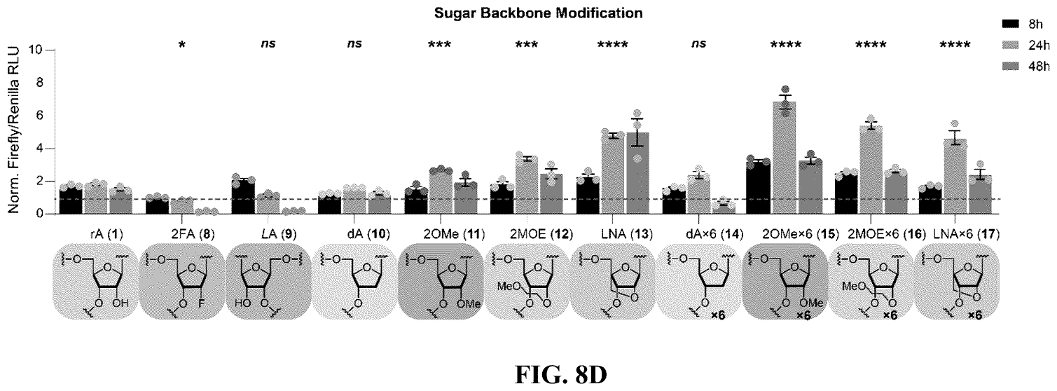

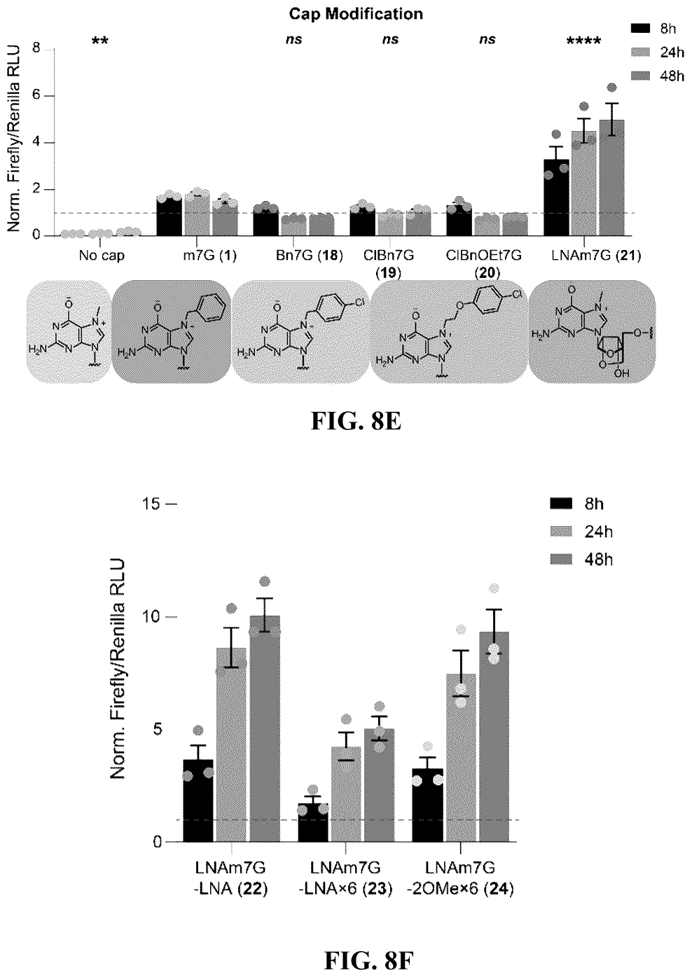

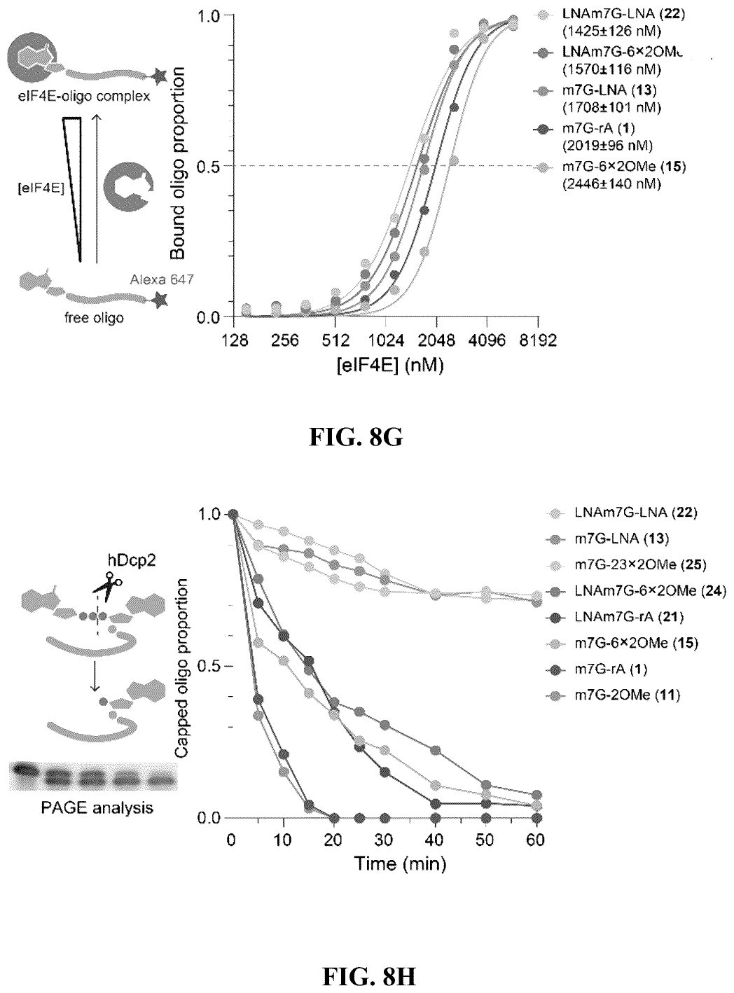

The modular nature of this workflow divergently constructs and evaluates mRNA with an array of cap and 5′ UTR modifications (FIG. 8A). mRNA structures were categorized into four dimensions: (1) first base identity, (2) phosphodiester linkages, (3) sugar backbone, and (4) cap modifications. First, the effects of nucleotide identity at the “+1 position” on translation, where protein yield varied in the order of A>G˜C˜U in accordance with literature reports, were evaluated (Sikorski et al., 2020, Nucleic Acids Res. 48: 1607-1626). Changing A to m⁶A further enhanced total protein production as expected, and, interestingly, incorporation of non-canonical inosine (I) showed similar effects (FIG. 8B). Subsequently used m⁷G-G, the “wild-type” base identities in ARCA-capped synthetic mRNAs as the benchmark for subsequent screening and m⁷G-A as the control construct for other aspects of mRNA modification.

In terms of phosphodiester linkage, while introduction of phosphorothioate (PS) onto the cap triphosphate bridge has previously been reported to boost protein yield (Kawaguchi et al., 2020, Angew. Chem. Int. Ed Engl. 59: 17403-17407), introduction of PS between +1 and +2 positions lowered translation. Further introduction of PS onto +1 to +7 positions rescued translation to normal levels but was still not beneficial (FIG. 8C).

Regarding modification on the ribose sugar backbone, replacing the adenosine 2′-hydroxyl to 2′-deoxy fluoro (2FA) hampered translation. Switching to chirality-inverted L-adenosine (LA) or 2′-deoxyadenosine (dA) resulted in non-significant changes in expression. Introduction of locked nucleic acid (LNA), 2′-methoxy (2OMe), and 2-methoxyethoxy (2MOE) noticeably increased mRNA translation, with introduction of a single LNA base led to a 4.8-fold increase. Expanding the dA backbone onto positions +1 to +6 did not significantly alter expression, while increasing the number of 2OMe and 2MOE modified bases resulted in 6.9-fold and 5.4-fold increase at 24 hrs, respectively. LNA, however, increased expression only at the +1 position, while modifying positions +1 to +6 led to decrease in activity. (FIG. 8D)

For evaluation of cap modifications, we synthesized m⁷GDP-Im analogues of cap structures previously reported to enhance translation. Cap structures were modified by replacing the m⁷G methyl group with benzyl (Bn⁷G) and chlorobenzyl (ClBn⁷G) or bearing LNA sugar backbone (m⁷G-LNA) (Kore et al., 2009, J. Am. Chem. Soc. 131: 6364-6365; Wojcik et al., 2021, Pharmaceutics 13(11): 1941). We also included chlorobenzyl-O-ethoxy (ClBnOEt⁷G), a structure previously developed not as an mRNA cap, but as a high-affinity eIF4E inhibitor (Chen et al., 2012, J. Med. Chem. 55: 3837-3851). Contrary to previous reports, all the aromatic substitutions did not show better performance than m⁷G, though all successfully triggered translation compared to uncapped mRNA. This discrepancy was possibly due to the fact that these hydrophobic modifications lead to better isolation of capped mRNA during purification after cotranscriptional capping, where we also observed a larger retention time shift on HPLC, in accordance with a recent report (Inagaki et al., 2023, Nat. Commun. 14: 2657). m⁷G-LNA indeed successfully enhanced translation by 4.5-fold at 24 hrs (FIG. 8E). We then demonstrated that these modifications from different dimensions could be combined to further amplify the effects where simultaneous introduction of m⁷G-LNA+LNA at +1 base or 2OMe on +1 to +6 further enhanced translation to 8.6-fold and 7.5-fold, respectively (FIG. 8F). Furthermore, chemical modification of the 5′ cap and downstream nucleotides can be combinatorially optimized to maximize affinity towards eIF4E or increase resistance to decapping by hDcp2 to potentially alter mRNA translation (FIG. 8G-H).

Internal Capping Drives Robust Translation on circRNA

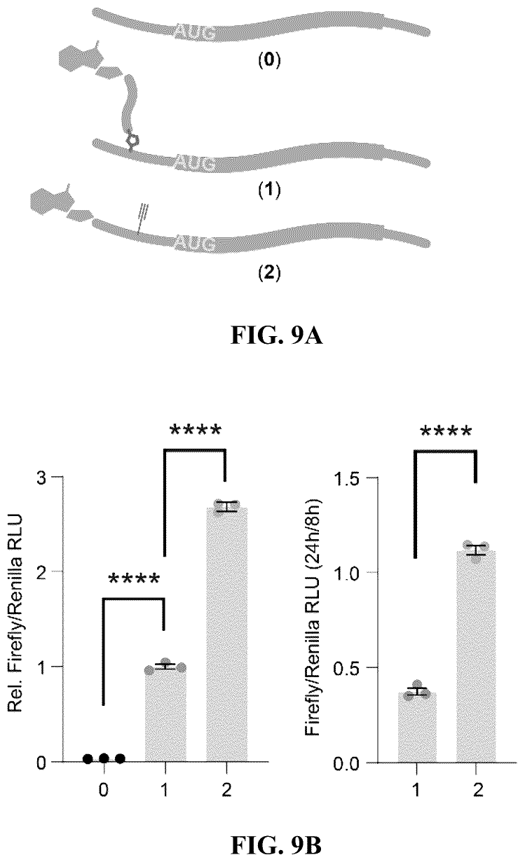

Because an additional internal cap structure enhanced translation on linear transcripts, we sought to apply this approach to drive translation of circRNAs. Conventionally, circRNAs lack a cap and poly(A) tail, requiring an IRES for translation initiation (FIG. 10A). Rate of initiation by IRES, however, is known to be slower than the canonical cap-dependent mechanism (Koch et al., 2020, Nat. Struct. Mol. Biol. 27: 1095-1104). While the branched cap could not prevent exonuclease degradation of uncapped mRNA “stem” (FIG. 9B), circRNAs have previously been reported to possess enhanced exonuclease resistance and stability in vivo (Wesselhoeft et al., 2018, Nat. Commun. 9: 2629; Chen et al., 2022, Nat. Biotechnol. 41(2): 262-272). An internal capping strategy might therefore simultaneously maintain the high stability of circRNA while hijacking the cap-dependent translation initiation mechanism to enhance its translatability (FIG. 10B and FIG. 10C). We named such capped-circular mRNA as QRNA, due to the resemblance of the construct to the letter “Q”.

In terms of the synthesis of QRNA, simultaneously achieving RNA circularization and incorporating a site-specific click-chemistry handle is a major challenge. As an initial proof of concept, we synthesized a minimal RNA encoding HiBiT and engineered its sequence such that it only contained a single cytosine in its UTR, allowing us to introduce an azide handle by replacing CTP with azide-labeled CTP (5-Azido-PEG4-CTP) during IVT. This minimal mRNA was then circularized by T4 RNA ligase with assistance by homology regions in the 5′ and 3′ UTRs to yield an azide-labeled circRNA, which was HPLC purified and confirmed to be RNase R-resistant. An m⁷G-capped, OU-labeled oligo was then conjugated azide-circRNA to yield a minimal QRNA (FIG. 4A-C). Encouragingly, enhanced translation was observed in QRNA compared to its circRNA precursor (FIG. 4D-4F).

To generalize QRNA synthesis to longer transcripts, an alternative workflow to nucleotide depletion is required. To this end, we synthesized a Nano luciferase (Nluc) encoding mRNA and circularized it through intron back-splicing, the standard approach for circRNA preparation. For incorporation of click-chemistry handles, a minimal hairpin sequence was introduced upstream of the CDS that could be site-specifically recognized and labeled by tRNA guanine transglycosylase (TGT) using pre-queuosine 1 (preQ1) (Ehret et al., 2018, Mol. Pharm. 15: 737-742). Utilizing TGT and synthetic preQ1-azide, we introduced a single azide handle onto the circRNA and conjugated it with a 5′ capped oligo containing a single 5-Octadiynyl dU site (OU) (FIG. 10D). To enrich QRNA abundance, a hydrophobic Bn⁷G cap analog was used to enhance its interaction on RP-HPLC, and the enriched product was characterized by a double RNase H assay (FIG. 10E). Although still not as efficient as regular m⁷G-capped linear mRNA, QRNA translated more effectively than its circRNA counterparts (with and without IRES) (FIG. 10F). On the other hand, as synthesis of the circRNA precursor still rely on ribozyme splicing, which is incompatible with m¹Ψ, QRNA was similarly only compatible with uridine, while linear mRNA expression was significantly boosted after m¹Ψ replacement (FIG. 10G). Such differences were not due to the inherent translatability of the QRNA construct but toxicity of uridine as the expression of cotransfected control Fluc mRNA was also significantly reduced when uridine-containing linear/QRNA were transfected (FIG. 10G). Consequently, only the multi-capped linear mRNA was carried on to animal experiments as it holds higher therapeutic value due to its compatibility with m¹Ψ.

### Materials and Methods

Plasmid Cloning, Characterization, and Purification (linear+circ): The mRNA expression vectors were generated as described before. Briefly, the protein of interests coding sequences (CDS) were inserted into an optimized backbone containing (in order) an T7 promoter sequence, a 5′ human alpha globin UTR, a CDS, a 3′ human alpha globin UTR, a 100×A template-encoded poly(A) tail, and an Esp3I linearization site. The CDS-containing plasmid/gene blocks were PCR amplified, gel-purified, and assembled into the optimized backbone using NEBuilder HiFi DNA Assembly Master Mix (NEB, E2621S), transformed into stable cells, and sequence-verified with whole plasmid sequencing.

The firefly luciferase construct was obtained from pmirGLO Dual-Luciferase miRNA Target Expression Vector (Promega, E1330). Renilla luciferase constructs were obtained from pmirGLO without cloning into the optimized vector. The nano luciferase constructs were obtained by gene synthesis from Genewiz.

Linear mRNA Synthesis and Characterization: DNA plasmids were obtained aforementioned and linearized by Esp3I (NEB, R0734S). Linearized plasmids are purified with the DNA Clean & Concentrator-25 kit from Zymo Research (D4033) and characterized with agarose gel electrophoresis. mRNA constructs were synthesized by in vitro transcription (IVT) using HiScribe T7 High Yield RNA Synthesis Kit [NEB, E2040S](for T7 promoter constructs) per manufacturer's protocol except 100% replacement of UTP with N1-methyl pseudouridine-5′-triphosphate [Trilink, N-1081-1] and addition of 1:50 SUPERase-In RNase inhibitor [ThermoFisher Scientific, AM2694]. Following IVT reaction, DNA templates were digested by TURBO DNase and purified using Monarch RNA cleanup kit [NEB, T2040L]. mRNA concentrations were quantified using the Qubit RNA HS Assay [ThermoFisher Scientific, Q32852] or the Qubit RNA BR Assay [ThermoFisher Scientific, Q10210]. Unless otherwise specified, mRNA products are suspended in 1:50 (v/v) RNase inhibitor-containing RNase-free water (subsequently referred to as RNase-free water) and stored at −80° C.

General conditions for RP-HPLC purification: All purification was conducted on an Agilent 1260 Infinity II HPLC. Acetonitrile (solvent A) [Sigma Aldrich, 34851], 100 mM hexylamine/acetic acid in water (pH 7.0, with 20% acetonitrile w/v) (solvent B), 50 mM diethylamine/acetic acid+50 mM ammonium acetate in water (pH 7.0) (solvent C) were used as the mobile phases and PLRP-S column as the stationary phase.
Method 1: 100 A pore size was used, 0% A+100% B (0-5 mins, hold); 10% A+90% B (5˜10 mins, linear increase); 25% A+75% B (10˜55 mins, linear increase).
Method 2: 4000 A pore size was used, 0% A+100% B (0 mins); 20% A+80% B (0˜2 mins, linear increase); 70% A+30% B (2˜30 mins, linear increase).
Method 3: 4000 A pore size was used, 0% A+100% C (0 mins); 25% A+75% B (0˜25 mins, linear increase).

Capped oligonucleotide synthesis: 12 nmol of solid phase synthesized oligonucleotide (with ammonium as counterion) was dissolved in a solution of 40 mM m7GDP-Im (or corresponding cap analogue) in 42 μL of anhydrous DMSO, and 8 μL of 1-methyl-imidazole was added. The reaction was mixed well and heated at 55° C. for 3 hrs. The reaction was then quenched by addition of 50 μL of water and directly subjected to HPLC purification using method 1. Fractions containing the capped products were pooled, lyophilized, and resuspended in RNase-free water and stored at −80° C. until being used. Concentrations of capped oligos were quantified using Qubit microRNA assay kit [Invitrogen, Q32880] and nanodrop.

Enzymatic Ligation of Modified Oligonucleotides to mRNAs: 5′-triphosphorylated mRNA was first treated with RppH [NEB, M0356S] per manufacturer's protocol to generate 5P-mRNA and purified with the Monarch RNA cleanup kit. Synthetic oligo and 5P-mRNA were mixed at a molar ratio of 25:1, and diluted in 2×50% PEG-8000, 10×T4 RNA ligase buffer, 10× T4 RNA ligase [Promega, M1051], and RNase-free water. The reaction was incubated at 37° C. for 30 mins and inactivated by the addition of 50×500 mM EDTA (pH 8.0). Products were purified first by the Monarch RNA cleanup kit and then by RNase-free HPLC (method 2). Purified fractions were pulled and desalted using the Monarch RNA cleanup kit and ligation efficiency was characterized using RNase H assay as described before. (17) In case of incomplete ligation, a second round of reaction was performed.

Modification Screening with Time-course DualLuciferase Assay: HeLa cells [ATCC, CCL-2] were maintained in DMEM culture media [ThermoFisher Scientific, 119951] containing 10% FBS and 1% penicillin-streptomycin [ThermoFisher Scientific, 15070063] in a 37° C. incubator with 5% CO₂ and passaged at a ratio of 1:10 every 3 days. On the day before mRNA transfection, HeLa cells were seeded at 90% confluence in individual wells on 24-well plates. The following day, 50 ng of Renilla luciferase (internal control) mRNA and 50 ng of modified Firefly luciferase mRNA were transfected using Lipofectamine MessengerMAX Transfection Reagent [ThermoFisher Scientific, LMRNA003] per manufacturer's protocols. Additional controls that contain only Renilla luciferase mRNA or lipofection reagent only were included. Three individual transfections were conducted for each condition. 6 hours after transfection, the transfection media was removed, and cells were trypsinized and reseeded to three white clear-bottom 96-well plates [Corning, 3610] in phenol-red-free media. At 8/24/48 hrs post transfection, cell culture media was removed and cells were rinsed with DPBS. Cells were lysed and luciferase activity was measured using the Promega Dual Glo Luciferase Assay System [Promega, E2920]. Briefly, 50 μL of PBS and 50 μL of Firefly luciferase working solution (prepared with manufacturer's protocols) were added to each well using multichannel pipette and mixed by pipetting. After 10 mins incubation with gentle shaking and protection from light at room temperature, Firefly luciferase luminescence was measured using a microplate reader. 50 μL of freshly prepared Renilla luciferase Stop&Glow working solution (prepared with manufacturer's protocols) was then added. Renilla luciferase luminescence was measured similarly after 10 mins incubation. For both Firefly and Renilla luminescence, background was measured by cells treated with only lipofectamine reagent and subtracted. Firefly luminescence/Renilla luminescence for each well was used as mRNA activity readout. In cases where Nluc and Fluc were used, the protocol was conducted similarly using the Nano-Glo Dual-Luciferase Reporter Assay System [Promega, N1610].

Circular mRNA Synthesis and Characterization: DNA templates were cloned as mentioned in previous sections, PCR amplified and gel purified to be used as IVT templates. CircRNA were synthesized as described in literature by using the HiScribe T7 High Yield RNA Synthesis Kit [NEB, E2040S]. Post IVT, DNA templates were digested with Turbo DNase [ThermoFisher, AM2238]. The reaction mixture was heated to 70° C. for 5 min and then immediately cooled on ice for 3 min, after which GTP was added to a final concentration of 2 mM, and the reaction mixture was incubated at 55° C. for 15 min. CircRNA was enriched by treatment with RNase R [Lucigen Corporation, RNR07250] for 1.5 hr, and the products were column purified. CircRNA products were characterized by gel electrophoresis.

QRNA Synthesis and Characterization: CircRNA bearing TGT hairpin was synthesized as described in previous sections. TGT enzyme was expressed in E Coli as described in the literature.(18) To label the circRNA with preQ1-azide, 1 μM of circRNA, 10 μM of preQ1-azide, 10 μM of TGT, 10 μL of SUPERase-In RNase inhibitor were incubated in 1×TGT reaction buffer (100 mM HEPES, pH 7.3, 5 mM DTT, and 20 mM MgCl₂) in a total of 100 μL reaction at 37° C. for 2 hours. The labeled circRNA was purified and subjected to click reaction with Bn⁷G-capped alkyne labeled oligo using the general condition for click reaction for 30 mins. The circRNA was then subjected to RP-HPLC purification to remove the linearized portions (method 2), pooled and desalted, and subjected to another round of RP-HPLC purification to isolate the QRNA product. QRNA product was characterized by RNase H assay with 2 primers upstream/downstream the TGT site.

Those of skill in the art will recognize or be able to ascertain using no more than routine experimentation, many equivalents to the specific embodiments of the invention described herein. Such equivalents are intended to be encompassed by the following claims.

TABLE 2
Sequences described in this application.

| Name | Sequence | SEQ ID NO | Note |
|---|---|---|---|
| FLAG | DYKDDDDK | 1 | |
| peptide | | | |
| 3xFLAG | DYKDDDDKDYKDDDDKDYKDDDD | 2 | |
| | K | | |
| Oligo 3.1 | AAGAGCGAGCTCTAATACGACTCA | 3 | U = N1-methylpseudouridine |
| | CTATAGGGGCCGCACTCGCCGGTC | | or uridine |
| | CCAAGCCCGGATAAAATGGGAGG | | |
| | GGGCGGGAAACCGCCTAACCATG | | |
| | CCGAGTGCGGCCGCAAAAAAGCA | | |
| | GACTGTAAATCTGAAAAAAGAGA | | |
| | AUAAACUAGUAUUCUUCUGGUCC | | |
| | CCACAGACUCAGAGAGAACCCGC | | |
| | CACCAUGGACUACAAAGACGAUG | | |
| | ACGACAAGAUGGACUACAAAGAC | | |
| | GAUGACGACAAGAUGGACUACAA | | |
| | AGACGAUGACGACAAGUAGAAAA | | |
| | AAAAAAAAAAAAAAAAAAAAAAA | | |
| | AAAGTGGCCGCGGTCGGCGTGGA | | |
| | CTGTAGAACACTGCCAATGCCGGT | | |
| | CCCAAGCCCGGATAAAAGTGGAG | | |
| | GGTACAGTCCACGCTCTAGATATA | | |
| | GCCTTATAGCCT | | |
| Oligo 3.4 | AGAAACAAAACAAAACAACCAAC | 4 | AG = 5′-cap analogue; U = 5- |
| | AACAACACAACAAU | | TCO-PEG4-2-deoxyuridine; U = |
| | | | N1-methylpseudouridine or |
| | | | uridine |
| HiBit tag | MVSGWRLFKKIS | 5 | |
| RNA | GGAAAAAAAAAAGAAAAAAAAGA | 6 | Scaffold for “circular” coding |
| scaffold | AUAAAUUUCUAUUAUUAAUAUAA | | portion of the tested mRNA. |
| containing a | UUAAAUUAAAAUUAAGAGAAGGG | | The underlined 5′ and 3′ |
| 5′ C-azide | AAGAUGGUGAGUGGAUGGAGAU | | regions were complementary |
| site | UAUUUAAGAAGAUUAGUUAGUA | | and facilitated enzymatic |
| | AUUUUAAUUUAAUUAUAUUAAUA | | circularization of the RNA by |
| | AUAGAAAUUUAAAAAAAAAAAAG | | enzymatic ligation. The bold |
| | AAAAAAAAAAAAAAAAAAAAAAA | | text shows the HiBit tag- |
| | A | | coding sequence. Additionally, |
| | | | the single C site (shown in bold |
| | | | italic text) was encoded by this |
| | | | template and was located in |
| | | | either the 5′ or 3′ |
| | | | complementary region. A site- |
| | | | specific azide functionalization |
| | | | in the circular RNA scaffold |
| | | | was achieved by only encoding |
| | | | a single cytidine in these |
| | | | templates (FIG. 4A). U = N1- |
| | | | methylpseudouridine |
| RNA | GGAAAAAAAAAAGAAAAAAAAGA | 7 | Same as SEQ ID NO: 6 above |
| scaffold | AUAAAUUUGUAUUAUUAAUAUAA | | |
| containing a | UUAAAUUAAAAUUAAGAGAAGGG | | |
| 3′ C-azide | AAGAUGGUGAGUGGAUGGAGAU | | |
| site | UAUUUAAGAAGAUUAGUUAGUA | | |
| | AUUUUAAUUUAAUUAUAUUAAUA | | |
| | AUACAAAUUUAAAAAAAAAAAAG | | |
| | AAAAAAAAAAAAAAAAAAAAAAA | | |
| | AA | | |
| RNase H | GCTAGCTCCAGGGTGTGGC | 8 | |
| primer | | | |
| upstream | | | |
| RNase H | GGGACCAGAAGAATACTAGTTTAT | 9 | |
| primer | TCGAT | | |
| downstream | | | |
| circRNA | GGGCGAATTGGAGCTCGGGAATTC | 10 | Circular RNA containing an |
| (iHRV) | TAGAGAAAATTTCGTCTGGATTAG | | engineered iHRV IRES. The |
| | TTACTTATCGTGTAAAATCTGATA | | sequence include the upstream |
| | AATGGAATTGGTTCTACATAAATG | | and downstream introns and |
| | CCTAACGACTATCCCTTTGGGGAG | | written from 5′ to 3′. T = |
| | TAGGGTCAAGTGACTCGAAACGAT | | uridine |
| | AGACAACTTGCTTTAACAAGTTGG | | |
| | AGATATAGTCTGCTCTGCATGGTG | | |
| | ACATGCAGCTGGATATAATTCCGG | | |
| | GGTAAGATTAACGACCTTATCTGA | | |
| | ACATAACGCTACCGTTTAATATTG | | |
| | CGTCATATAAAAAAAAAAAACCA | | |
| | AAAAAAAAAACAAAAAAAAAAAA | | |
| | TAATTGACTAATATCTTAAAACAG | | |
| | CGGATGGGTACCCCACCATCCGAC | | |
| | CCACTGGGTGTAGTACTCTGGTAC | | |
| | TTCGTACCTTTGTACGCCTGTTCTT | | |
| | CCCATTGTACCCTTCCTGAACTTCC | | |
| | AACCCAAGTAACGTTAGAAGCTCA | | |
| | ACATTTAGTACAACAGGAAGCACC | | |
| | ACATCCAGTGGTGTTTAGTACAAG | | |
| | CACTTCTGTTTCCCCGGAGCGAGG | | |
| | TATAGGCTGTACCCACTGCCAAAA | | |
| | ACCTTTAACCGTTATCCGCCAACC | | |
| | AACTACGTAAAAGCTAGTAGTATT | | |
| | ATGTTTTTAACTAGGCGTTCGATC | | |
| | AGGTGGATTTCCCCTCCACTAGTT | | |
| | TGGTCGATGAGGCTAGGAATTCCC | | |
| | CACGGGTGACCGTGTCCTAGCCTG | | |
| | CGTGGCGGCCAACCCAGCCCACTC | | |
| | ACTATTTGTTTTCGCGCCCAGTTGC | | |
| | AAAAAGTGTCGGGGCTGGGACGC | | |
| | CTTTTTATAGACATGGTGTGAAGA | | |
| | CTCGCATGTGCTTGGTTGTGATTCC | | |
| | TCCGGCCCCTGAATGCGGCTAACC | | |
| | TTAACCCTGGAGCCTTGTGTCACA | | |
| | AACCAGTGATGATAAGGTCGTAAT | | |
| | GAGCAATTCCGGGACGGGACCGA | | |
| | CTACTTTGGGTGTCCGTGTTTCTTA | | |
| | TTTTTCTTATTATTGTCTTATGGTC | | |
| | ACAGCATATATATAACATATACTG | | |
| | TGATCATGGATGGTCTTCACACTC | | |
| | GAAGATTTCGTTGGGGACTGGCGA | | |
| | CAGACAGCCGGCTACAACCTGGAC | | |
| | CAAGTCCTTGAACAGGGAGGTGTG | | |
| | TCCAGTTTGTTTCAGAATCTCGGG | | |
| | GTGTCCGTAACTCCGATCCAAAGG | | |
| | ATTGTCCTGAGCGGTGAAAATGGG | | |
| | CTGAAGATCGACATCCATGTCATC | | |
| | ATCCCGTATGAAGGTCTGAGCGGC | | |
| | GACCAAATGGGCCAGATCGAAAA | | |
| | AATTTTTAAGGTGGTGTACCCTGT | | |
| | GGATGATCATCACTTTAAGGTGAT | | |
| | CCTGCACTATGGCACACTGGTAAT | | |
| | CGACGGGGTTACGCCGAACATGAT | | |
| | CGACTATTTCGGACGGCCGTATGA | | |
| | AGGCATCGCCGTGTTCGACGGCAA | | |
| | AAAGATCACTGTAACAGGGACCCT | | |
| | GTGGAACGGCAACAAAATTATCG | | |
| | ACGAGCGCCTGATCAACCCCGACG | | |
| | GCTCCCTGCTGTTCCGAGTAACCA | | |
| | TCAACGGAGTGACCGGCTGGCGGC | | |
| | TGTGCGAACGCATTCTGGCGTAGT | | |
| | AGTCTCGAGCTGGTACTGCATGCA | | |
| | CGCAATGCTAGCTGCCCCTTTCCC | | |
| | GTCCTGGGTACCCCGAGTCTCCCC | | |
| | CGACCTCGGGTCCCAGGTATGCTC | | |
| | CCACCTCCACCTGCCCCACTCACC | | |
| | ACCTCTGCTAGTTCCAGACACCTC | | |
| | CCAAGCACGCAGCAATGCAGCTCA | | |
| | AAACGCTTAGCCTAGCCACACCCC | | |
| | CACGGGAAACAGCAGTGATTAAC | | |
| | CTTTAGCAATAAACGAAAGTTTAA | | |
| | CTAAGCTATACTAACCCCAGGGTT | | |
| | GGTCAATTTCGTGCCAGCCACACC | | |
| | CTGGAGCTAGCCTCAGTAGATGTT | | |
| | TTCTTGGGTTAATTGAGGCCTGAG | | |
| | TATAAGGTGACTTATACTTGTAAT | | |
| | CTATCTAAACGGGGAACCTCTCTA | | |
| | GTAGACAATCCCGTGCTAAATTGT | | |
| | AGGACTGCCCTTTAATAAATACTT | | |
| | CTATATTTAAAGAGGTATTTATGA | | |
| | AAAGCGGAATTTATCAGATTAAAA | | |
| | ATACTTTCTCTAGAGTCGACCTGC | | |
| | AGG | | |
| circRNA | ACTTGCTTTAACAAGTTGGAGATA | 11 | Circular RNA. The sequence |
| (globin) | TAGTCTGCTCTGCATGGTGACATG | | include the upstream and |
| | CAGCTGGATATAATTCCGGGGTAA | | downstream introns and |
| | GATTAACGACCTTATCTGAACATA | | written from 5′ to 3′. T = |
| | ACGCTACCGTTTAATATTGCGTCA | | uridine |
| | TATAAAAAAAAAAACCAAAAAAA | | |
| | AAAACAAAAAAAAAAATAATTGA | | |
| | CTAATTAAGCAGACTGTAAATCTG | | |
| | CTAAAATCGAATAAACTAGTATTC | | |
| | TTCTGGTCCCCACAGACTCAGAGA | | |
| | GAACCCGCCACCATGGTCTTCACA | | |
| | CTCGAAGATTTCGTTGGGGACTGG | | |
| | CGACAGACAGCCGGCTACAACCTG | | |
| | GACCAAGTCCTTGAACAGGGAGGT | | |
| | GTGTCCAGTTTGTTTCAGAATCTC | | |
| | GGGGTGTCCGTAACTCCGATCCAA | | |
| | AGGATTGTCCTGAGCGGTGAAAAT | | |
| | GGGCTGAAGATCGACATCCATGTC | | |
| | ATCATCCCGTATGAAGGTCTGAGC | | |
| | GGCGACCAAATGGGCCAGATCGA | | |
| | AAAAATTTTTAAGGTGGTGTACCC | | |
| | TGTGGATGATCATCACTTTAAGGT | | |
| | GATCCTGCACTATGGCACACTGGT | | |
| | AATCGACGGGGTTACGCCGAACAT | | |
| | GATCGACTATTTCGGACGGCCGTA | | |
| | TGAAGGCATCGCCGTGTTCGACGG | | |
| | CAAAAAGATCACTGTAACAGGGA | | |
| | CCCTGTGGAACGGCAACAAAATTA | | |
| | TCGACGAGCGCCTGATCAACCCCG | | |
| | ACGGCTCCCTGCTGTTCCGAGTAA | | |
| | CCATCAACGGAGTGACCGGCTGGC | | |
| | GGCTGTGCGAACGCATTCTGGCGT | | |
| | AGTAGTCTCGAGCTGGTACTGCAT | | |
| | GCACGCAATGCTAGCTGCCCCTTT | | |
| | CCCGTCCTGGGTACCCCGAGTCTC | | |
| | CCCCGACCTCGGGTCCCAGGTATG | | |
| | CTCCCACCTCCACCTGCCCCACTC | | |
| | ACCACCTCTGCTAGTTCCAGACAC | | |
| | CTCCCAAGCACGCAGCAATGCAGC | | |
| | TCAAAACGCTTAGCCTAGCCACAC | | |
| | CCCCACGGGAAACAGCAGTGATTA | | |
| | ACCTTTAGCAATAAACGAAAGTTT | | |
| | AACTAAGCTATACTAACCCCAGGG | | |
| | TTGGTCAATTTCGTGCCAGCCACA | | |
| | CCCTGGAGCTAGCAAAAAAAAAA | | |
| | AAAAAAAAAAAAAAAAAAAAAAA | | |
| | AAAAAACTCAGTAGATGTTTTCTT | | |
| | GGGTTAATTGAGGCCTGAGTATAA | | |
| | GGTGACTTATACTTGTAATCTATCT | | |
| | AAACGGGGAACCTCTCTAGTAGAC | | |
| | AATCCCGTGCTAAATTGTAGGACT | | |
| | GCCCTTTAATAAATACTTCTATATT | | |
| | TAAAGAGGTATTTATGAAAAGCGG | | |
| | AATTTATCAGATTAAAAATACTTT | | |
| | CTCTAGAGTCGACCTGCAG | | |
| QRNA | ACTTGCTTTAACAAGTTGGAGATA | 12 | Capped circular RNA. The |
| (globin) | TAGTCTGCTCTGCATGGTGACATG | | sequence include the upstream |
| | CAGCTGGATATAATTCCGGGGTAA | | and downstream introns and |
| | GATTAACGACCTTATCTGAACATA | | written from 5′ to 3′ and |
| | ACGCTACCGTTTAATATTGCGTCA | | branched on the TGT hairpin |
| | TATAAAAAAAAAAACCAAAAAAA | | (bold). Cap structure Bn7G. T = |
| | AAAACAAAAAAAAAAATAATTGA | | uridine |
| | CTAATTAAGCAGACTGTAAATCT | | |
| | GCTAAAATCGAATAAACTAGTATT | | |
| | CTTCTGGTCCCCACAGACTCAGAG | | |
| | AGAACCCGCCACCATGGTCTTCAC | | |
| | ACTCGAAGATTTCGTTGGGGACTG | | |
| | GCGACAGACAGCCGGCTACAACCT | | |
| | GGACCAAGTCCTTGAACAGGGAG | | |
| | GTGTGTCCAGTTTGTTTCAGAATCT | | |
| | CGGGGTGTCCGTAACTCCGATCCA | | |
| | AAGGATTGTCCTGAGCGGTGAAAA | | |
| | TGGGCTGAAGATCGACATCCATGT | | |
| | CATCATCCCGTATGAAGGTCTGAG | | |
| | CGGCGACCAAATGGGCCAGATCG | | |
| | AAAAAATTTTTAAGGTGGTGTACC | | |
| | CTGTGGATGATCATCACTTTAAGG | | |
| | TGATCCTGCACTATGGCACACTGG | | |
| | TAATCGACGGGGTTACGCCGAACA | | |
| | TGATCGACTATTTCGGACGGCCGT | | |
| | ATGAAGGCATCGCCGTGTTCGACG | | |
| | GCAAAAAGATCACTGTAACAGGG | | |
| | ACCCTGTGGAACGGCAACAAAATT | | |
| | ATCGACGAGCGCCTGATCAACCCC | | |
| | GACGGCTCCCTGCTGTTCCGAGTA | | |
| | ACCATCAACGGAGTGACCGGCTGG | | |
| | CGGCTGTGCGAACGCATTCTGGCG | | |
| | TAGTAGTCTCGAGCTGGTACTGCA | | |
| | TGCACGCAATGCTAGCTGCCCCTT | | |
| | TCCCGTCCTGGGTACCCCGAGTCT | | |
| | CCCCCGACCTCGGGTCCCAGGTAT | | |
| | GCTCCCACCTCCACCTGCCCCACT | | |
| | CACCACCTCTGCTAGTTCCAGACA | | |
| | CCTCCCAAGCACGCAGCAATGCAG | | |
| | CTCAAAACGCTTAGCCTAGCCACA | | |
| | CCCCCACGGGAAACAGCAGTGATT | | |
| | AACCTTTAGCAATAAACGAAAGTT | | |
| | TAACTAAGCTATACTAACCCCAGG | | |
| | GTTGGTCAATTTCGTGCCAGCCAC | | |
| | ACCCTGGAGCTAGCAAAAAAAAA | | |
| | AAAAAAAAAAAAAAAAAAAAAAA | | |
| | AAAAAAACTCAGTAGATGTTTTCT | | |
| | TGGGTTAATTGAGGCCTGAGTATA | | |
| | AGGTGACTTATACTTGTAATCTAT | | |
| | CTAAACGGGGAACCTCTCTAGTAG | | |
| | ACAATCCCGTGCTAAATTGTAGGA | | |
| | CTGCCCTTTAATAAATACTTCTATA | | |
| | TTTAAAGAGGTATTTATGAAAAGC | | |
| | GGAATTTATCAGATTAAAAATACT | | |
| | TTCTCTAGAGTCGACCTGCAG | | |
| mRNA | AGGAATAAACTAGTATTCTTCTGG | 13 | Linear mRNA with the same |
| (globin, U) | TCCCCACAGACTCAGAGAGAACCC | | sequence and UTR as above |
| | GCCACCATGGGATCCGTCTTCACA | | but cotranscriptionally capped |
| | CTCGAAGATTTCGTTGGGGACTGG | | and tailed; the mRNA contains |
| | CGACAGACAGCCGGCTACAACCTG | | wild-type uridine. Cap |
| | GACCAAGTCCTTGAACAGGGAGGT | | structure m7G |
| | GTGTCCAGTTTGTTTCAGAATCTC | | |
| | GGGGTGTCCGTAACTCCGATCCAA | | |
| | AGGATTGTCCTGAGCGGTGAAAAT | | |
| | GGGCTGAAGATCGACATCCATGTC | | |
| | ATCATCCCGTATGAAGGTCTGAGC | | |
| | GGCGACCAAATGGGCCAGATCGA | | |
| | AAAAATTTTTAAGGTGGTGTACCC | | |
| | TGTGGATGATCATCACTTTAAGGT | | |
| | GATCCTGCACTATGGCACACTGGT | | |
| | AATCGACGGGGTTACGCCGAACAT | | |
| | GATCGACTATTTCGGACGGCCGTA | | |
| | TGAAGGCATCGCCGTGTTCGACGG | | |
| | CAAAAAGATCACTGTAACAGGGA | | |
| | CCCTGTGGAACGGCAACAAAATTA | | |
| | TCGACGAGCGCCTGATCAACCCCG | | |
| | ACGGCTCCCTGCTGTTCCGAGTAA | | |
| | CCATCAACGGAGTGACCGGCTGGC | | |
| | GGCTGTGCGAACGCATTCTGGCGT | | |
| | AGTGATAATAGCTCGAGGCTGGAG | | |
| | CCTCGGTGGCCATGCTTCTTGCCC | | |
| | CTTGGGCCTCCCCCCAGCCCCTCC | | |
| | TCCCCTTCCTGCACCCGTACCCCC | | |
| | GTGGTCTTTGAATAAAGTCTGAGT | | |
| | GGGCGGCACAATTGAAAAAAAAA | | |
| | AAAAAAAAAAAAAAAAAAAAAAA | | |
| | AGCATATGACTAAAAAAAAAAAA | | |
| | AAAAAAAAAAAAAAAAAAAAAAA | | |
| | AAAAAAAAAAAAAAAAAAAAAAA | | |
| | AA | | |
| mRNA | AGGAATAAACTAGTATTCTTCTGG | 14 | Linear mRNA with the same |
| (globin, | TCCCCACAGACTCAGAGAGAACCC | | sequence and UTR as above |
| | GCCACCATGGGATCCGTCTTCACA | | but cotranscriptionally capped |
| | CTCGAAGATTTCGTTGGGGACTGG | | and tailed; the mRNA contains |
| | CGACAGACAGCCGGCTACAACCTG | | m1Y′. Cap structure m7G. |
| | GACCAAGTCCTTGAACAGGGAGGT | | |
| | GTGTCCAGTTTGTTTCAGAATCTC | | |
| | GGGGTGTCCGTAACTCCGATCCAA | | |
| | AGGATTGTCCTGAGCGGTGAAAAT | | |
| | GGGCTGAAGATCGACATCCATGTC | | |
| | ATCATCCCGTATGAAGGTCTGAGC | | |
| | GGCGACCAAATGGGCCAGATCGA | | |
| | AAAAATTTTTAAGGTGGTGTACCC | | |
| | TGTGGATGATCATCACTTTAAGGT | | |
| | GATCCTGCACTATGGCACACTGGT | | |
| | AATCGACGGGGTTACGCCGAACAT | | |
| | GATCGACTATTTCGGACGGCCGTA | | |
| | TGAAGGCATCGCCGTGTTCGACGG | | |
| | CAAAAAGATCACTGTAACAGGGA | | |
| | CCCTGTGGAACGGCAACAAAATTA | | |
| | TCGACGAGCGCCTGATCAACCCCG | | |
| | ACGGCTCCCTGCTGTTCCGAGTAA | | |
| | CCATCAACGGAGTGACCGGCTGGC | | |
| | GGCTGTGCGAACGCATTCTGGCGT | | |
| | AGTGATAATAGCTCGAGGCTGGAG | | |
| | CCTCGGTGGCCATGCTTCTTGCCC | | |
| | CTTGGGCCTCCCCCCAGCCCCTCC | | |
| | TCCCCTTCCTGCACCCGTACCCCC | | |
| | GTGGTCTTTGAATAAAGTCTGAGT | | |
| | GGGCGGCACAATTGAAAAAAAAA | | |
| | AAAAAAAAAAAAAAAAAAAAAAA | | |
| | AGCATATGACTAAAAAAAAAAAA | | |
| | AAAAAAAAAAAAAAAAAAAAAAA | | |
| | AAAAAAAAAAAAAAAAAAAAAAA | | |
| | AA | | |

1. A type 2 capped circular RNA molecule, comprising:
an mRNA encoding a peptide or polypeptide;
a 5′ end containing a cap structure;
a derivatized nucleotide located between the cap molecule and the mRNA region encoding the polypeptide; and
a 3′ end covalently linked to the derivatized nucleotide.

2. A type 1 capped circular RNA molecule, comprising:
an RNA oligonucleotide comprising a 5′ end containing a cap structure and a 3′ end moiety;
a circular RNA molecule comprising an mRNA encoding a peptide or polypeptide, and a derivatized nucleotide located within the circular RNA molecule,
wherein the 3′ end moiety of the oligonucleotide is covalently linked to the derivatized nucleotide on the circular RNA molecule.

3. A type 3 capped circular RNA molecule, comprising:
an RNA oligonucleotide comprising a 5′ end containing a cap structure and a 3′ end moiety; and
a circular RNA molecule comprising a twister ribozyme, an mRNA encoding a peptide or polypeptide, an oligonucleotide portion that forms a hairpin, and a derivatized nucleotide located within the hairpin,
wherein the 3′ end moiety of the oligonucleotide is covalently linked to the derivatized nucleotide within the hairpin of the circular RNA molecule.

4. The capped circular RNA molecule of claim 1, wherein the derivatized nucleotide comprises a moiety that can react with the 3′ end moiety by bioconjugation chemistry.

5. (canceled)

6. The capped circular RNA molecule of claim 1, wherein the cap structure comprises
(a) 7-methylguanosine (m⁷G), 7-benzylguanosine (Bn⁷G), 7-chlorobenzylguanosine (ClBn⁷G), chlorobenzyl-O-ethoxyguanosine (ClBnOEt⁷G), or any derivative thereof; or
(b) a 7-methylguanosine cap further comprising one or more Locked Nucleic Acid (LNA), or one or more 2′-methoxy (2OMe), or any derivative thereof.

7. (canceled)

8. The capped circular RNA molecule of claim 1, further comprising one or more modified nucleotides.

9. The capped circular RNA molecule of claim 8, wherein the modified nucleotides comprise:
(a) pseudouridine, N¹-methylpseudouridine (m¹Ψ), 6-methyladenosine (m⁶A), 5-methylcytidine, inosine, or any derivatives thereof; or
(b) locked nucleic acid (LNA), 2′-methoxyribose (2-OMe), 2-methoxyehthoxy (2-MOE) sugar backbone, or any derivatives thereof.

10-19.

20. A method of producing a capped circular RNA molecule according to claim 1, comprising:
a) synthesizing an RNA oligonucleotide comprising a 5′ end containing a cap structure, an mRNA encoding a peptide or polypeptide, a derivatized nucleotide located between the cap structure and the mRNA region encoding the polypeptide, and a 3′ end containing moiety; and
b) reacting the derivatized nucleotide with the 3′ end moiety to form the covalently linked capped circular RNA molecule.

21. (canceled)

22. A method of producing a capped circular RNA molecule according to claim 3, wherein synthesis of the RNA oligonucleotide comprises:
a) synthesizing a first RNA oligonucleotide primer comprising the 5′ end containing a cap structure, the derivatized nucleotide, and a complementary sequence to a DNA template encoding a peptide or a polypeptide;
b) transcribing the first RNA oligonucleotide from the primer along the DNA template to produce an mRNA encoding a peptide or a polypeptide;
c) synthesizing a second RNA oligonucleotide comprising a 3′ end containing a moiety;
d) ligating the 3′ end of the first RNA oligonucleotide encoding the peptide or polypeptide sequence with the 5′ end of the second RNA molecule.

23. A method of producing a capped circular RNA molecule according to claim 2, comprising:
a) producing a circularized RNA molecule comprising an mRNA region encoding a peptide or polypeptide, and a derivatized nucleotide outside the mRNA region;
b) synthesizing an RNA oligonucleotide comprising a 5′ end containing cap structure and a 3′ end containing a moiety reactive with the derivatized nucleotide; and
c) reacting the derivatized nucleotide with the 3′ end moiety of the RNA oligonucleotide form a covalently link between the RNA oligonucleotide and the circular RNA.

24-33. (canceled)

34. The capped circular RNA molecule of claim 2, wherein the derivatized nucleotide comprises a moiety that can react with the 3′ end moiety by bioconjugation chemistry.

35. The capped circular RNA molecule of claim 2, wherein the cap structure comprises:
(a) 7-methylguanosine (m⁷G), 7-benzylguanosine (Bn⁷G), 7-chlorobenzylguanosine (ClBn⁷G), chlorobenzyl-O-ethoxyguanosine (ClBnOEt⁷G), or any derivative thereof; or
(b) a 7-methylguanosine cap further comprising one or more Locked Nucleic Acid (LNA), or one or more 2′-methoxy (2OMe), or any derivative thereof.

36. The capped circular RNA molecule of claim 2, further comprising one or more modified nucleotides.

37. The capped circular RNA molecule of claim 36, wherein the modified nucleotides comprise:
(a) pseudouridine, N¹-methylpseudouridine (m¹Ψ), 6-methyladenosine (m⁶A), 5-methylcytidine, inosine, or any derivatives thereof; or
(b) locked nucleic acid (LNA), 2′-methoxyribose (2-OMe), 2-methoxyehthoxy (2-MOE) sugar backbone, or any derivatives thereof.

38. The capped circular RNA molecule of claim 2, wherein the circular RNA comprises a plurality of mRNA regions encoding one or a plurality of polypeptides.

39. The capped circular RNA molecule of claim 38, further comprising:
a plurality of RNA oligonucleotides comprising a 5′ end containing the cap structure and a 3′ end moiety; and
a plurality of derivatized nucleotides at a position in the circular RNA 5′ to each of the mRNA regions encoding a peptide or polypeptide,
wherein each 3′ end of each of the plurality of the RNA oligonucleotides is covalently linked to each of the plurality of the derivatized nucleotides.

40. The capped circular RNA molecule of claim 39, wherein each mRNA region encoding the peptide or the polypeptide comprises a 3′ polyA sequence.

41. The capped circular RNA molecule of claim 40, wherein the one or plurality of polypeptides encode Cas9, base editors, or derivatives.

42. The capped circular RNA molecule of claim 40, wherein the one or plurality of polypeptides include therapeutic proteins.

43. The capped circular RNA molecule of claim 3, wherein the derivatized nucleotide comprises a moiety that can react with the 3′ end moiety by bioconjugation chemistry.

44. The capped circular RNA molecule of claim 3, wherein the cap structure comprises:
(a) 7-methylguanosine (m⁷G), 7-benzylguanosine (Bn⁷G), 7-chlorobenzylguanosine (ClBn⁷G), chlorobenzyl-O-ethoxyguanosine (ClBnOEt⁷G), or any derivative thereof; or
(b) a 7-methylguanosine cap further comprising one or more Locked Nucleic Acid (LNA), or one or more 2′-methoxy (2OMe), or any derivative thereof.

45. The capped circular RNA molecule of claim 3, further comprising one or more modified nucleotides.

46. The capped circular RNA molecule of claim 45, wherein the modified nucleotides comprise 6-methyladenosine (m⁶A), 5-methylcytidine, inosine, or any derivatives thereof.

47. The capped circular RNA molecule of claim 3, wherein the circular RNA comprises a plurality of mRNA regions encoding one or a plurality of polypeptides.

48. The capped circular RNA molecule of claim 47, further comprising:
a plurality of RNA oligonucleotides comprising a 5′ end containing the cap structure and a 3′ end moiety; and
a plurality of derivatized nucleotides at a position in the circular RNA 5′ to each of the mRNA regions encoding a peptide or polypeptide,
wherein each 3′ end of each of the plurality of the RNA oligonucleotides is covalently linked to each of the plurality of the derivatized nucleotides.

49. The capped circular RNA molecule of claim 48, wherein each mRNA region encoding the peptide or the polypeptide comprises a 3′ polyA sequence.

50. The capped circular RNA molecule of claim 49, wherein the one or plurality of polypeptides encode Cas9, base editors, or derivatives.

51. The capped circular RNA molecule of claim 49, wherein the one or plurality of polypeptides include therapeutic proteins.


SEQUENCE LISTING (14 sequences)

SEQ ID NO: 1 - protein, 8 aa, synthetic construct
DYKDDDDK

SEQ ID NO: 2 - protein, 24 aa, synthetic construct
DYKDDDDKDYKDDDDKDYKDDDDK

SEQ ID NO: 3 - RNA, 407 nt, synthetic construct
  source 1..407: mol_type = other RNA
  modified_base 1..407: mod_base = OTHER; note = n is N1-methylpseudouridine or uridine
aagagcgagctctaatacgactcactataggggccgcactcgccggtcccaagcccggataaaatgggagggggcgggaaaccgcctaaccatgccgagtgcggccgcaaaaaagcagactgtaaatctgaaaaaagagaanaaacnagnanncnncnggnccccacagacncagagagaacccgccaccanggacnacaaagacgangacgacaaganggacnacaaagacgangacgacaaganggacnacaaagacgangacgacaagnagaaaaaaaaaaaaaaaaaaaaaaaaaaaaaagtggccgcggtcggcgtggactgtagaacactgccaatgccggtcccaagcccggataaaagtggagggtacagtccacgctctagatatagccttatagcct

SEQ ID NO: 4 - RNA, 37 nt, synthetic construct
  source 1..37: mol_type = other RNA
  modified_base 37: mod_base = OTHER; note = TCO-PEG4-2-deoxyuridine
  modified_base 1..2: mod_base = OTHER; note = m7G(5')ppp(5')(2'OMeA)pG
  modified_base 37: mod_base = OTHER; note = N1-methylpseudouridine or uridine
agaaacaaaacaaaacaaccaacaacaacacaacaat

SEQ ID NO: 5 - protein, 12 aa, synthetic construct
MVSGWRLFKKIS

SEQ ID NO: 6 - RNA, 183 nt, synthetic construct
  source 1..183: mol_type = other RNA
  modified_base 1..183: mod_base = OTHER; note = n is N1-methylpseudouridine
ggaaaaaaaaaagaaaaaaaagaanaaannncnannannaananaannaaannaaaannaagagaagggaaganggngagngganggagannannnaagaagannagnnagnaannnnaannnaannanannaanaanagaaannnaaaaaaaaaaaagaaaaaaaaaaaaaaaaaaaaaaaa

SEQ ID NO: 7 - RNA, 184 nt, synthetic construct
  source 1..184: mol_type = other RNA
  modified_base 1..184: mod_base = OTHER; note = n is N1-methylpseudouridine
ggaaaaaaaaaagaaaaaaaagaanaaannngnannannaananaannaaannaaaannaagagaagggaaganggngagngganggagannannnaagaagannagnnagnaannnnaannnaannanannaanaanacaaannnaaaaaaaaaaaagaaaaaaaaaaaaaaaaaaaaaaaaa

SEQ ID NO: 8 - RNA, 19 nt, synthetic construct
  source 1..19: mol_type = other RNA
gctagctccagggtgtggc

SEQ ID NO: 9 - RNA, 29 nt, synthetic construct
  source 1..29: mol_type = other RNA
gggaccagaagaatactagtttattcgat

SEQ ID NO: 10 - RNA, 2018 nt, synthetic construct
  source 1..2018: mol_type = other RNA
gggcgaattggagctcgggaattctagagaaaatttcgtctggattagttacttatcgtgtaaaatctgataaatggaattggttctacataaatgcctaacgactatccctttggggagtagggtcaagtgactcgaaacgatagacaacttgctttaacaagttggagatatagtctgctctgcatggtgacatgcagctggatataattccggggtaagattaacgaccttatctgaacataacgctaccgtttaatattgcgtcatataaaaaaaaaaaaccaaaaaaaaaaacaaaaaaaaaaaataattgactaatatcttaaaacagcggatgggtaccccaccatccgacccactgggtgtagtactctggtacttcgtacctttgtacgcctgttcttcccattgtacccttcctgaacttccaacccaagtaacgttagaagctcaacatttagtacaacaggaagcaccacatccagtggtgtttagtacaagcacttctgtttccccggagcgaggtataggctgtacccactgccaaaaacctttaaccgttatccgccaaccaactacgtaaaagctagtagtattatgtttttaactaggcgttcgatcaggtggatttcccctccactagtttggtcgatgaggctaggaattccccacgggtgaccgtgtcctagcctgcgtggcggccaacccagcccactcactatttgttttcgcgcccagttgcaaaaagtgtcggggctgggacgcctttttatagacatggtgtgaagactcgcatgtgcttggttgtgattcctccggcccctgaatgcggctaaccttaaccctggagccttgtgtcacaaaccagtgatgataaggtcgtaatgagcaattccgggacgggaccgactactttgggtgtccgtgtttcttatttttcttattattgtcttatggtcacagcatatatataacatatactgtgatcatggatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtagtagtctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcctcagtagatgttttcttgggttaattgaggcctgagtataaggtgacttatacttgtaatctatctaaacggggaacctctctagtagacaatcccgtgctaaattgtaggactgccctttaataaatacttctatatttaaagaggtatttatgaaaagcggaatttatcagattaaaaatactttctctagagtcgacctgcagg

SEQ ID NO: 11 - RNA, 1311 nt, synthetic construct
  source 1..1311: mol_type = other RNA
acttgctttaacaagttggagatatagtctgctctgcatggtgacatgcagctggatataattccggggtaagattaacgaccttatctgaacataacgctaccgtttaatattgcgtcatataaaaaaaaaaaccaaaaaaaaaaacaaaaaaaaaaataattgactaattaagcagactgtaaatctgctaaaatcgaataaactagtattcttctggtccccacagactcagagagaacccgccaccatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtagtagtctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaaaaaaaaaaactcagtagatgttttcttgggttaattgaggcctgagtataaggtgacttatacttgtaatctatctaaacggggaacctctctagtagacaatcccgtgctaaattgtaggactgccctttaataaatacttctatatttaaagaggtatttatgaaaagcggaatttatcagattaaaaatactttctctagagtcgacctgcag

SEQ ID NO: 12 - RNA, 1311 nt, synthetic construct; identical to SEQ ID NO: 11
  source 1..1311: mol_type = other RNA

SEQ ID NO: 13 - RNA, 811 nt, synthetic construct
  source 1..811: mol_type = other RNA
aggaataaactagtattcttctggtccccacagactcagagagaacccgccaccatgggatccgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtagtgataatagctcgaggctggagcctcggtggccatgcttcttgccccttgggcctccccccagcccctcctccccttcctgcacccgtacccccgtggtctttgaataaagtctgagtgggcggcacaattgaaaaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaa

SEQ ID NO: 14 - RNA, 811 nt, synthetic construct; identical to SEQ ID NO: 13
  source 1..811: mol_type = other RNA
  modified_base 1..811: mod_base = OTHER; note = T is N1-methylpseudouridine or uridine